(19) United States
(12) Patent Application Publication
(10) Pub. No.: US 2003/0176653 A1
(43) Pub. Date: Sep. 18, 2003

(54) Orally immunogenic bacterial enterotoxins expressed in transgenic plants

(75) Inventors: Hugh S. Mason, Phoenix, AZ (US); Charles J. Arntzen, Superstition Mountain, AZ (US)

Correspondence Address: PALMER & DODGE, LLP, KATHLEEN M. WILLIAMS, 111 HUNTINGTON AVENUE, BOSTON, MA, 02199, US

(73) Assignee: Boyce Thompson Institute

(21) Appl. No.: 10/334,729

(22) Filed: Dec. 30, 2002

Related U.S. Application Data
(63) Continuation of application No. 09/470,124, filed on Dec. 22, 1999, now abandoned.
(60) Provisional application No. 60/113,507, filed on Dec. 22, 1998.

(51) Int. Cl. C07K 1/00; C07K 14/00; C07K 17/00

(52) U.S. Cl. 530/350

(57) ABSTRACT

The invention provides mutant Escherichia coli heat labile (LT) and Vibrio cholerae toxin (CT) polypeptides and the polynucleotides that encode them. The mutant LT and CT polypeptides can be readily produced in plants and can be used to treat or prevent diseases caused by E. coli and V. cholera. The polypeptides are also useful as adjuvants.

### RELATED APPLICATION

This application claims the benefit of U.S. application Ser. No. 60/113,507; which is incorporated in its entirety by reference.

### TECHNICAL FIELD

The present invention is related to genetic engineering of plants and the transformation of plants using recombinant DNA techniques to produce edible vaccines. More particularly, the invention relates to the production of oral vaccines and adjuvants in transgenic plants using polynucleotides encoding E. coli heat-labile toxin subunits LT-A and LT-B, cholera toxin subunits CT-A and CT-B, and mutants of thereof.

### BACKGROUND OF THE INVENTION

Vaccines are administered to humans and animals to induce an immune response against viruses, bacteria, and other types of pathogenic organisms. In the economically advanced countries of the world, vaccines have brought many diseases under control. However, many vaccines for such diseases as poliomyelitis, measles, mumps, rabies, foot and mouth are too expensive for use in lesser developed countries.

Because of the simplicity of oral delivery, there is great interest in discovering new oral vaccine technology. Appropriately delivered oral immunogens can stimulate humoral, mucosal, and cellular immunity and have the potential to provide cost-effective, safe vaccines for use in developing countries or inner cities where large-scale parenteral immunization is not practical or is extremely difficult to implement. Such vaccines may be based upon bacterial or viral vector systems that express epitopes from diverse pathogens (multivalent vaccines) or may be based upon purified antigens delivered singularly or in combination with other relevant antigens.

The majority of human pathogens initiate disease by interaction with mucosal surfaces. Bacterial and viral pathogens that act through this mechanism first make contact with the mucosal surface where they can attach and colonize, or may be taken up by specialized absorptive cells (M cells) in the epithelium that overlay Peyer's patches and other lymphoid follicles. Organisms that enter the lymphoid tissues may be readily killed within the lymphoid follicles, thereby provoking a potentially protective immunological response as antigens are delivered to immune cells within the follicles (e.g., Vibrio cholerae). Alternatively, pathogenic organisms capable of surviving local defense mechanisms may spread from the follicles and subsequently cause local or systemic disease (e.g., Salmonella spp., and poliovirus in immunocompromised hosts).

Secretory IgA (sIgA) antibodies directed against specific virulence determinants of infecting organisms play an important role in overall mucosal immunity. In many cases, it is possible to prevent the initial infection of mucosal surfaces by stimulating production of mucosal sIgA levels directed against relevant virulence determinants of an infecting organism. Thus, secretary IgA may prevent the initial interaction of the pathogen with the mucosal surface by blocking attachment and/or colonization, neutralizing surface acting toxins, or preventing invasion of the host cells.

Parenterally administered inactivated whole-cell and whole-virus preparations are effective at eliciting protective serum IgG and delayed type hypersensitivity reactions against organisms that have a significant serum phase in their pathogenesis (e.g., Salmonella typhi, Hepatitis B). However, parenteral vaccines are not effective at eliciting mucosal sIgA responses and are ineffective against bacteria that interact with mucosal surfaces but do not invade (e.g., Vibrio cholerae).

Oral immunization can be effective for induction of specific sIgA responses if the antigens are presented to the T and B lymphocytes and accessory cells contained within the Peyer's patches where preferential IgA B-cell development is initiated. The Peyer's patches contain helper T(TH)-cells that mediate B-cell isotype switching directly from IgM cells to IgA. B-cells then migrate to the mesenteric lymph nodes and undergo differentiation, enter the thoracic duct, the general circulation, and subsequently seed all of the secretary tissues of the body, including the lamina propria of the gut and respiratory tract. IgA is then produced by the mature plasma cells, complexed with membrane-bound secretary component, and transported onto the mucosal surface where it is available to interact with invading pathogens. This common mucosal immune system is a basis for use of live oral vaccines and oral immunization for protection against pathogenic organisms that initiate infection by first interacting with mucosal surfaces.

Enteric bacterial diseases such as cholera, dysentery, Escherichia coli related diarrheas, and typhoid fever are major causes of morbidity and mortality, especially in developing countries where sanitation conditions are less than adequate. Several types of vaccines against these organisms have been developed and tested over the years, among them killed whole cells, subunits of toxins, and live attenuated bacteria administered parenterally or orally. The majority of the morbidity and mortality due to bacterial diarrheal disease result from infection with V. cholerae and the cholera-related enterotoxic organisms (i.e., E. coli that produce cholera-like enterotoxin).

Strains of E. coli cause diarrheal disease by a variety of mechanisms, including production of one or more enterotoxins. One of these toxins, referred to as the heat-labile enterotoxin (LT), is immunologically and physicochemically related to the cholera enterotoxin. Spangler, Microbiol. Rev. 56:622-647 (1992).

The high cost of production and purification of synthetic peptides manufactured by chemical or fermentation based processes can prevent their broad scale use as oral vaccines. The production of immunogenic proteins in transgenic plants, on the other hand, offers an economical alternative. Attempts have been made to produce transgenic plants expressing bacterial antigens of E. coli and Streptococcus mutans. For instance, Curtiss et al. (WO 90/0248) demonstrated the transformation of sunflower with an LT-B gene. Also, the expression of LT-B and its assembly into GI-binding pentamers in tobacco and potato plants has been reported. Haq et al., Science. 268:714-716 (1995). Additionally, Amtzen et al (WO 96/12801) disclose vectors for the independent and coordinate expression of LT-A and LT-B, which optionally contain a SEKDEL microsomal retention signal. The transformation of tobacco and potato with these genes is described. Mice and chickens were fed extracts or raw tubers of the transgenic plants and serum and mucosal immune responses were exhibited to LT. Potatoes have been transformed with a plant-optimized synthetic gene encoding the native LT-B subunit with its 21 residue bacterial signal peptide. Mice fed slices of the transgenic potato were observed to have elevated levels of serum and mucosal anti-LT-B although none were completely protected. Mason et al., Vaccine. 16:1336-1343 (1998).

It has been shown that the inclusion of KDEL amino acid sequences at the carboxy terminus of a protein can enhance the recognition for that protein by the plant ER retention machinery (see, e.g., Munro et al., Cell. 48: 988-997 (1987). However, such modifications can be problematic because other factors, such as protein conformation or protein folding in the transformed cells, may interfere with the availability of this carboxy terminus signal to the plant ER retention machinery. Retention of key biological properties in the recombinant proteins produced in plants, specifically ligand binding and the presentation of antigenic epitopes, is of considerable importance to the successful production of edible vaccines in transgenic plants. Fortunately, relatively low amounts of the proteins may be required, since their effects are amplified by the immune system.

Oral vaccines derived from transgenic plants offer the potential to be an effective and inexpensive means for inducing immune responses, including secretory immune responses, to enterotoxins in animals including humans. Previous transgenic plant systems transformed with the native LT-A, LT-B, or their CT counterparts, typically show stunting of the plants. For example, CT-A has been expressed in tobacco plants, and reportedly caused spontaneous leaf lesions, induced pathogenesis-related proteins, and high levels of salicylic acid. Beffa et al. EMBO J. 14:5753 (1995). Thus, the enzymatic activity of CT-A and LT-A, in which ADP-ribosylates heterotrimeric GTP-binding proteins, appears to be active in plant cells and causes toxicity symptoms. A need exists for proven techniques that yield healthy transgenic plants and plant seeds that can induce a desired immune response without significant side effects to the plants. The proteins expressed by such plants should have substantially reduced toxic properties while retaining their immunogenicity and/or adjuvanticity. The present invention is directed to new approaches to achieving these objectives.

### SUMMARY OF THE INVENTION

It is an object of the invention to provide mutant Escherichia coli heat labile (LT) and Vibrio cholerae toxin (CT) polypeptides and the polynucleotides that encode them. It is a further object of the invention to provide the mutant LT and CT polypeptides in plant cells that can be used to treat or prevent diseases caused by E. coli and V. cholera. It is another object of the invention to provide polypeptides that are useful as adjuvants. These and other objects of the invention are provided by one or more of the embodiments described below.

One embodiment of the invention provides a polynucleotide comprising a nucleic acid sequence encoding a mutant Escherichia coli heat-labile toxin (LT) A subunit (LT-A) or a mutant cholera toxin (CT) A subunit (CT-A). The mutant LT-A or CT-A polypeptide has reduced enzyme activity as compared to a wild-type LT-A or CT-A polypeptide and has at least one codon of the polynucleotide altered to a plant-preferred codon.

In another embodiment of the invention the polynucleotide further comprises a nucleic acid sequence encoding a LT B subunit (LT-B) or a CT B subunit (CT-B).

In still another embodiment of the invention the polynucleotide comprising a nucleic acid sequence encoding LT-B or CT-B comprises at least one altered codon. The altered codon is a plant preferred codon.

In yet another embodiment of the invention the polynucleotide comprising a nucleic acid sequence encoding LT-B is shown in SEQ ID NO:46.

In even another embodiment of the invention the polynucleotide comprising a nucleic acid sequence encoding CT-B is shown in SEQ ID NO:48.

In another embodiment of the invention the polynucleotide-comprising a nucleic acid sequence encoding LT-B or CT-B is operably linked to a promoter.

In still another embodiment of the invention a polynucleotide comprising a nucleic acid sequence encoding LT-B or CT-B further comprises a tobacco etch virus (TEV)-5′ untranslated region, and/or a microsomal retention signal sequence such as a C-terminal SEKDEL (SEQ ID NO:59) sequence.

In yet another embodiment of the invention a polynucleotide is provided that encodes LT-A or CT-A, wherein the LT-A or CT-A polypeptide has reduced ADP-ribosylation activity as compared to wild-type LT-A or CT-A.

In even another embodiment of the invention a polynucleotide is provided that encodes a mutant LT-A or CT-A polypeptide which has reduced enzyme activity as compared to a wild-type LT-A or CT-A polypeptide. At least one codon of the polynucleotide is altered to a plant-preferred codon. The polynucleotide encodes a single amino acid mutant LT-A or CT-A polypeptide or a double amino acid mutant LT-A or CT-A polypeptide.

In another embodiment of the invention a polynucleotide is provided that encodes a mutant LT-A or CT-A polypeptide which has reduced enzyme activity as compared to a wild-type LT-A or CT-A polypeptide. At least one codon of the polynucleotide is altered to a plant-preferred codon. The polynucleotide encodes a mutant LT-A or CT-A polypeptide comprising a mutation at an amino acid selected from the group consisting of amino acids 61, 63, 72, 106, and 192.

In still another embodiment of the invention a polynucleotide is provided that encodes a mutant LT-A or CT-A polypeptide which has reduced enzyme activity as compared to a wild-type LT-A or CT-A polypeptide. At least one codon of the polynucleotide is altered to a plant-preferred codon. The mutation is selected from the group consisting of an amino acid substitution, an amino acid addition, an amino acid deletion, and a truncation.

In still another embodiment of the invention the mutation prevents cleavage of an A subunit into A1 and A2 fragments.

In yet another embodiment of the invention the polynucleotide is shown in a sequence listing selected from the sequence listings shown in SEQ ID NO:43, SEQ ID NO:44, and SEQ ID NO:45.

In even another embodiment of the invention the polynucleotide is operably linked to a plant promoter.

In another embodiment of the invention the polynucleotide further comprises a tobacco mosaic virus (TMV)-5′ untranslated region and/or at least one flanking T-DNA right border region of Agrobacterium.

In yet another embodiment of the invention an expression vector is provided. The expression vector comprises polynucleotide encoding a mutant LT-A or CT-A polypeptide that has reduced enzyme activity as compared to a wild-type LT-A or CT-A polypeptide and at least one codon of the polynucleotide is altered to a plant-preferred codon. The expression vector can further comprise a selectable marker, an E. coli origin of replication, and/or an Agrobacterium tumefaciens origin of replication. In even another embodiment of the invention an E. coli cell or an Agrobacterium tumefaciens transformed with the expression vector is provided The Agrobacterium tumefaciens cell can further comprise a helper Ti plasmid.

In another embodiment of the invention a transgenic plant cell comprising a polynucleotide encoding a mutant LT-A or CT-A polypeptide that has reduced enzyme activity as compared to a wild-type LT-A or CT-A polypeptide is provided. The polynucleotide has at least one codon altered to a plant-preferred codon.

In still another embodiment of the invention a transgenic plant seed is provided. The plant seed comprises a polynucleotide encoding a mutant LT-A or a mutant CT-A, wherein the mutant LT-A or CT-A polypeptide has reduced enzyme activity as compared to a wild-type LT-A or CT-A polypeptide.

In yet another embodiment of the invention a transgenic eukaryotic cell is provided. The cell comprises a polynucleotide encoding a mutant LT-A or a mutant CT-A, wherein the mutant LT-A or CT-A polypeptide has reduced enzyme activity as compared to a wild-type LT-A or CT-A polypeptide. The cell can be an insect cell or a plant cell. If the cell is a plant cell the polynucleotide can be integrated into the nuclear genome of the plant cell. Further, at least one codon of the polynucleotide can be altered to a plant-preferred codon. The plant cell can be selected from the group of plant cells consisting of tobacco, potato, tomato, carrot, and banana.

In even another embodiment of the invention the plant cell comprises a polynucleotide which further comprises a nucleic acid sequence encoding a LT-B subunit or a CT-B subunit. The polynucleotide comprising a nucleic acid sequence encoding LT-B or CT-B can comprise at least one altered codon, wherein the altered codon is a plant preferred codon. A polynucleotide comprising a nucleic acid sequence encoding LT-B is shown in SEQ ID NO:46 and SEQ ID NO:48. The polynucleotide can comprise a promoter, a tobacco etch virus (TEV)-5′ untranslated region, and/or a microsomal retention signal sequence, such as a C-terminal SEKDEL (SEQ ID NO:59) sequence.

In another embodiment of the invention a plant cell can comprise a polynucleotide wherein the LT-A or CT-A polypeptide encoded therein has reduced ADP-ribosylation activity as compared to wild-type LT-A or CT-A.

In still another embodiment of the invention the plant cell comprises a polynucleotide that encodes a single amino acid mutant of an LT-A or CT-A polypeptide or a double amino acid mutant of an LT-A or CT-A polypeptide.

In yet another embodiment of the invention the plant cell comprises a polynucleotide encoding a mutant LT-A or CT-A polypeptide. The polypeptide comprises a mutation at an amino acid selected from the group consisting of amino acids 61, 63, 72, 106, and 192. The mutation can be selected from the group consisting of an amino acid substitution, an amino acid addition, an amino acid deletion, and a truncation.

In even another embodiment of the invention the plant cell comprises a polynucleotide encoding a polypeptide comprising a mutation that prevents cleavage of an A subunit into A1 and A2 fragments.

In another embodiment of the invention the plant cell comprises a polynucleotide shown in SEQ ID NO:43, SEQ ID NO:44, and SEQ ID NO:45.

In still another embodiment of the invention the plant cell comprises a polynucleotide operably linked to a plant promoter.

In yet another embodiment of the invention an immunogenic composition is provided. The immunogenic composition comprises a plant cell. The plant cell comprises a polynucleotide encoding a mutant LT-A or a mutant CT-A, wherein the mutant LT-A or CT-A polypeptide has reduced enzyme activity as compared to a wild-type LT-A or CT-A polypeptide. The plant cell can be present in plant tissue selected from the group consisting of a fruit, leaf, tuber, plant organ, seed protoplast, and callus. The immunogenic composition can comprise juice or extract of the plant cell. The immunogenic composition can further comprise an adjuvant.

In even another embodiment of the invention a method of eliciting an immune response in an animal or human comprising the step of administering the immunogenic composition to a human or animal is provided. The composition can be administered orally, by for example consuming the transgenic plant cell.

In another embodiment of the invention a method of eliciting an immune response is provided by administering a polypeptide of the invention that has been purified from a plant cell. In still another embodiment of the invention the polypeptide is administered by a technique selected from the group consisting of intramuscular, oral, intradermal, intraperitoneal, subcutaneous, and intranasal. An adjuvant can also be administered.

In yet another embodiment of the invention an immune response is elicited by administering a polypeptide of the invention. The immune response is selected from the group of immune responses consisting of humoral; mucosal; cellular; humoral and mucosal; humoral and cellular; mucosal and cellular; and humoral, mucosal and cellular.

In even another embodiment of the invention a transgenic plant that expresses a mutant LT-A or CT-A polypeptide is provided. The rate of growth of the plant is same or similar to that of a plant that does not produce a LT or CT polypeptide. Further, the rate of growth of the plant can be greater than that of a plant that produces a wild-type LT-A or CT-A polypeptide.

In another embodiment of the invention the transgenic plant comprises the mutant LT-A or CT-A polypeptide which has reduced enzyme activity such as reduced ADP-ribosylating activity. In still another embodiment of the invention the transgenic plant further expresses an LT-B or CT-B polypeptide.

In yet another embodiment of the invention the transgenic plant is transformed with an expression vector of the invention.

In even another embodiment of the invention an adjuvant comprising a mutant LT-A or CT-A polypeptide is provided. The mutant LT-A or CT-A polypeptide has reduced enzyme activity, such as ADP ribosylating activity, as compared to a wild-type LT-A or CT-A polypeptide. The adjuvant can further comprise an LT-B or a CT-B polypeptide.

In another embodiment of the invention the polypeptide adjuvant is expressed by a eukaryotic cell transformed with a polynucleotide comprising a nucleic acid sequence encoding a mutant LT-A or mutant CT-A. At least one codon of the polynucleotide is altered to a plant-preferred codon. The cell can be a plant cell.

In still another embodiment of the invention the adjuvant is administered orally. The adjuvant can be administered separately from an immunogenic composition or concurrently with an immunogenic composition.

In yet another embodiment of the invention a polynucleotide encoding the mutant LT-A or CT-A polypeptide is fused to a polynucleotide encoding an antigen. The antigen can be selected from the group consisting of a colonization antigen, a virulence antigen, an epitope of a virulence antigen, and an epitope of a colonization antigen. The fused polynucleotide can be expressed in an eukaryotic cell, such as a plant cell.

As used herein, an “antigen” is a macromolecule capable of eliciting an immune response in a human or in an animal.

An “epitope” is a portion of an antigen that comprises the particular part of the antigen to which the antibody binds.

A “colonization antigen” or “virulence antigen” is an antigen of a pathogenic microorganism that is associated with the ability of the microorganism to colonize or invade its host.

A “polynucleotide,” “nucleic acid,” and the like is a polynucleotide that encodes a polypeptide. The polynucleotide or nucleic acid can include introns, marker genes, signal sequences, regulatory elements, such as promoters, enhancers and termination sequences, and the like.

An “expression vector” is a plasmid, such as pBR322, pUC, or ColE1; a virus such as an adenovirus, Sindbis virus, simian virus 40, alphavirus vectors, and cytomegalovirus and retroviral vectors, such as murine sarcoma virus, mouse mammary tumor virus, Moloney murine leukemia virus, and Rous sarcoma virus. Bacterial vectors, such as Salmonella ssp., Yersinia enterocolitica, Shigella spp., Vibrio cholerae, Mycobacterium strain BCG, and Listeria monocytogenes can be used. Minichromosomes such as MC and MC1, bacteriophages, virus particles, virus-like particles, cosmids (plasmids into which phage lambda cos sites have been inserted) and replicons (genetic elements that are capable of replication under their own control in a cell) can also be used as expression vectors. Preferably, an expression vector is capable of transforming eukaryotic cells, including, for example plant tissue.

A “foodstuff’ or “edible plant material” and the like is any plant material that can be directly ingested by animals or human as a nutritional source or dietary complement. An edible plant material includes a plant or any material obtained from a plant, which is suitable for ingestion by mammal or other animals including humans. This term is intended to include raw plant material that may be fed directly to animals or processed plant material that is fed to animals, including humans.

An “immune response” comprises the response of a host to an antigen. A humoral immune response comprises the production of antibodies in response to an antigen or antigens. A cellular immune response includes responses such as a helper T-cell (CD4⁺) response and a cytotoxic T-cell lymphocyte (CD8⁺) response. A mucosal immune response (or secretory immune response) comprises the production of secretory (sIgA) antibodies. An immune response can comprise one or a combination of these responses.

An “immunogenic agent” is an antigen or antigens capable of eliciting an immune response. Preferably, the immune response is elicited in a human or animal upon oral ingestion of a eukaryotically expressed antigen. An “immunogenic composition” contains one or more immunogenic agents, optionally in combination with a carrier, adjuvant, or the like.

A “fusion protein” is a protein containing at least 2, 3, 4, 5, 10, or more same or different amino acid sequences linked in a polypeptide where the sequences were not natively expressed as a single protein. Fusion proteins can be produced by well known genetic engineering techniques.

### BRIEF DESCRIPTION OF THE DRAWINGS

FIG. 1 depicts the plant-optimized nucleotide sequence encoding LT-A. Altered codons from the wild-type (i.e., plant preferred codons) are indicated by underlining. A GTG codon accommodates an NcoI restriction site embracing the initiator methionine codon. The codon at position 244 was altered to AAG to create the sLTA-K63 mutant.

FIG. 2 shows the forward and reverse oligomers used to assemble an LT-A gene adapted for expression in plants.

FIG. 3 illustrates the expression cassettes of various plasmids constructed according to the principles of the present invention. The DNA between the left border (LB) and right border (RB) is integrated stably into the plant cell nuclear genome in Agrobacterium-mediated transformation. An expression cassette for npt2 allows selection of transformed plant cells on media containing kanamycin. 5′ NOS: nopaline synthase promoter of Agrobacterium; NPT2: coding sequence of neomycin phosphotransferase; 3′ Ag7: 3′ flanking region from Agrobacterium gene 7; 5′ 35S: cauliflower mosaic virus 35S promoter having duplicated enhancer; sLTA, plant-optimized synthetic LT-A coding sequence, 3′ PIN2: 3′ flanking region from pin2 tomato proteinase inhibitor II gene; sLT-B: plant-optimized synthetic LT-B coding sequence; 5′ E8: promoter of the tomato E8 gene; 3′ VSP: 3′ flanking region of soybean vspb vegetative storage protein P gene; TEV 5′ UTR: tobacco etch virus RNA 5′ untranslated region; TMV 5′ UTR: tobacco mosaic virus RNA 5′ untranslated region. Selected restriction endonuclease sites are indicated.

FIG. 4A shows a plant-optimized CT-A nucleotide sequence and encoded amino acid sequence. The wild-type CT-A had two cryptic poly-A signal sequence, one cryptic 5′-intron splice recognition sequence, and 14 “CG” potential methylation sites. The plant-optimized sequence has no cryptic signal sequences and no CG sequences. FIG. 4B shows a plant-optimized CT-A-K63 mutant nucleotide sequence and encoded amino acid sequence. FIG. 4C shows a plant-optimized CT-A-R72 mutant nucleotide sequence and encoded amino acid sequence. FIG. 4D shows a plant-optimized CT-A-G192 mutant nucleotide sequence and encoded amino acid sequence.

FIG. 5 shows a plant-optimized LT-B gene (S) used in construction of pTH110 and the corresponding native bacterial LT-B gene (N). Nucleotides shown in bold in the S sequence were changed and the amino acid sequence is shown below the N sequence. The sequence encoding the mRNA destabilizing motif AUUUA in the native gene is underlined. Putative polyadenylation signals in the native gene are double underlined. The first amino acid of the mature LT-B protein (Ala) is indicated in bold underlined type. The first 21 residues, which constitute the bacterial signal peptide, are shown in italics. The second codon is changed from AAT encoding Asn to GTG encoding Val, in order to create a NcoI restriction site at the 5′ end. Selected restriction endonuclease sites are indicated.

FIG. 6 shows a nucleotide sequence encoding CT-B, which has been optimized for expression in plants. In the wild-type, one RNA polymerase II termination sequence was found and seven CG potential methylation sites were found. No cryptic signal sequences or CG sequences occur in the plant-optimized gene.

FIG. 7 shows a plasmid map of pNV110.

FIG. 8 shows a plasmid map of pHB117.

FIG. 9 shows a Western blot analysis of the SLT103 tomato fruit using two different antibodies: a mouse monoclonal that is specific for LT-A and a goat polyclonal antiserum that was raised against LT holotoxin.

FIG. 10 shows a plasmid map of pQEK63.

FIG. 11 shows a plasmid map of pHB306.

FIG. 12 shows a plasmid map of pvspSP-LTB.

FIG. 13 shows a plasmid map of pTHαS110.

FIG. 14 shows a plasmid map of pSLT407.

FIG. 15 shows a plasmid with a dicistronic cassette for coexpression of LT-A-R72 and LT-B with an IRES sequence upstream of the LT-B polynucleotide.

### DETAILED DESCRIPTION OF THE INVENTION

Immunogenic Polypeptides

Heat-labile toxin (LT) is an A-B type ADP-ribosylating toxin which has five identical 11.6 kDa B (binding) subunits that assemble into a doughnut shaped pentamer and one 27 kDa A (enzymatic) subunit which is cleaved into LT-A1 and LT-A2. Sixma et al., Nature. 351:371-377 (1991). The assembled LT toxin is referred to as an LT holotoxin. The B subunit (LT-B) mediates binding of the toxin to the ganglioside G_{MI} on the surface of gut mucosal cells. Orally administered LT-B can induce a serum IgG and mucosal IgA response in humans and animals and can be used as an adjuvant. The LT-A1 fragment is endocytosed and causes chloride secretion and consequent water loss from gut cells. LT-A1 has an enzymatic activity that mediates transfer of ADP-ribose to heterotrimeric GTP-binding proteins, thus locking on cyclic AMP production and causing excessive chloride secretion. It is this ADP-ribosyltransferase activity that is responsible for the toxicity associated with these and related toxin molecules. LT and mutants thereof can be produced by enterotoxigenic E. coli (ETEC) or by any other organism, including transgenic organisms, that express the genes for LT-A and LT-B or mutant genes thereof.

A nucleotide sequence encoding an LT-A subunit has been reported. Yamamoto et al., J. Biol. Chem. 259: 5037-5044 (1984). The deduced amino acid sequence of LT-A consists of 258 residues including a signal peptide of 18 residues. A nucleotide sequence encoding an LT-B subunit has also been reported. Yamamoto et al., (1983) J Bacteriol. 155: 728-733 (1983). This protein is initially synthesized as a 124 residue protein, which loses its signal peptide resulting in a mature 103 residue protein.

Cholera toxin (CT), is similar to LT and contains one A subunit and five identical B subunits. An assembled cholera toxin is referred to as a CT holotoxin. The CT-A subunit must be nicked to produce fragments A1 and A2 in order to become enzymatically active. The native B subunit of CT induces an immune response in animal and humans and can be used as an adjuvant. Bergquist et al., Infect. Immun. 65:2676-2684 (1997). CT and mutants thereof can be produced by Vibrio cholera or by any other organism, including transgenic organisms, that express the genes for CT-A (ctxa) and CT-B (ctxB) (Mekalanos et al., Nature. 306:551-7 (1983)) or mutant genes thereof.

LT and CT enterotoxins are structurally, functionally, and immunologically related to each other, and antibodies against either LT or CT react with both toxins (Spangler, 1992). Both LT and CT are potent mucosal immunogens, and have strong mucosal adjuvant qualities. Clements et al., Vaccine 6:269-277 (1988); Holmgren et al., Vaccine. 11:1179-1184 (1993). Thus, immune responses against other antigens can be enhanced by co-presentation with low doses of LT or CT.

Mutations in the A subunit of LT or CT can eliminate or reduce enzymatic activity, including ADP-ribosylation activity, of an A subunit as compared to a wild-type subunit. Without being bound to one particular theory, it is believed that the eliminated or reduced activity of an A subunit is related to reduced or eliminated toxicity upon expression of a mutant polypeptide in transgenic plants. Thus, LT and CT polypeptides comprising mutations in LT-A and CT-A subunits are more readily expressed in plants than the native subunit. Further, LT and CT holotoxins and subunits thereof containing such mutations are more readily expressed in plants, and accumulate to higher levels in plants than do wild-type holotoxins or wild-type subunits. Thus, enhanced immunity and adjuvanticity can be achieved with transgenic plants according to the present invention. Therefore, the invention comprises a mutation or mutations, i.e., amino acid substitutions, additions, deletions, truncations, or combinations thereof, in a LT-A, CT-A, LT, or CT polypeptide. Preferably, the mutation reduces or eliminates enzyme activity of the polypeptide compared to a wild-type polypeptide. Even more preferably, the mutation reduces or eliminates the ADP-ribosylation activity of the polypeptide compared to a wild-type polypeptide. As used herein, “reduces” refers to at least a 50% reduction in ADP-ribosylation activity, preferably 60%, 70%, 80% or 90% and even up to 100%; “Uminates” refers to no more than 5% ADP-ribosylation activity being detectabale in a preparation. ADP-ribosylation activity of polypeptides of the invention can be measured by, for example, the procedure of Collier and Kandel using wheat germ extract enriched in elongation factor 2. J. Biol. Chem. 246:1496-1503 (1971).

Mutations in the LT-A subunit that reduce or eliminate enzymatic activity, and correspondingly reduce toxicity in, for example plants, have been identified. Di Tommaso et al Infect. Immun. 64:974-979 (1996); Fontana et al, Infect. Immun. 63:2356-2360 (1995); Pizza et al., J. Exp. Med. 180: 2147-2153 (1994). A single amino acid substitution in the active site of LT-A (referred to as S63K or LT-K63) reduces the enzymatic activity (and thus toxicity) of LT by greater than 10⁶-fold while retaining adjuvant activity. The LT-K63 mutant has been used as an adjuvant in vaccinations of mice with H. pylori antigen. Ghiara et al. Infect. Immun. 65:4996-5002 (1997). Similarly, a single amino acid substitution, known as the R192G or LTG192 (Arg 192-Lys192) within the cleavage site of LT-A eliminates ADP-ribosyltransferase activity of LT, while retaining adjuvant activity. Dickinson & Clements, Infect. Imun. 63:1617-1623 (1995). Another LT mutant, designated LT-R72, has a single amino acid substitution in the active site of LT-A and has a reduced, but measurable enzymatic activity with retention of mucosal adjuvant activity. Giuliani et al., J. Exp. Med. 187: 1123-1132 (1998); Rappuoli, LTK63 and LTR72: Immunogens and mucosal adjuvants. Consultative WHO/NIH Meeting on the Evaluation of Vaccines Administered via Mucosal Surfaces, NIH, Bethesda, Md., Feb. 9, 1998. Two mutants of the CT toxin molecule (CT-K63 and CT-S106) have reduced ADP-ribosylation activities. Douce et al., Infect. Immun. 65:2821-2828 (1997). A single amino acid substitution in the active site of CT-A (S63 to K), known as CTK63, reduces the enzymatic activity (and thus toxicity) of CT while retaining immunologic activity (Fontana et al, 1995). Also, a mutant of the A subunit of CT (S61F) elicits an immune response in mice. Yamamoto, Proc. Natl. Acad. Sci. USA. 94: 5267-5272 (1997).

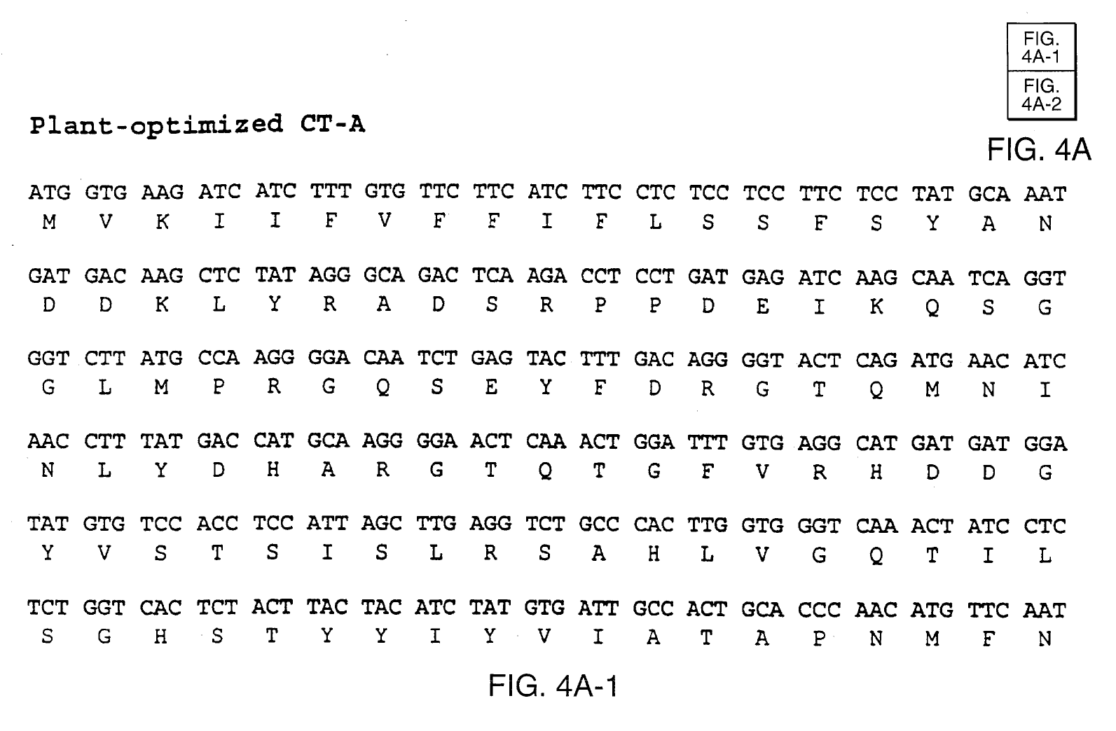

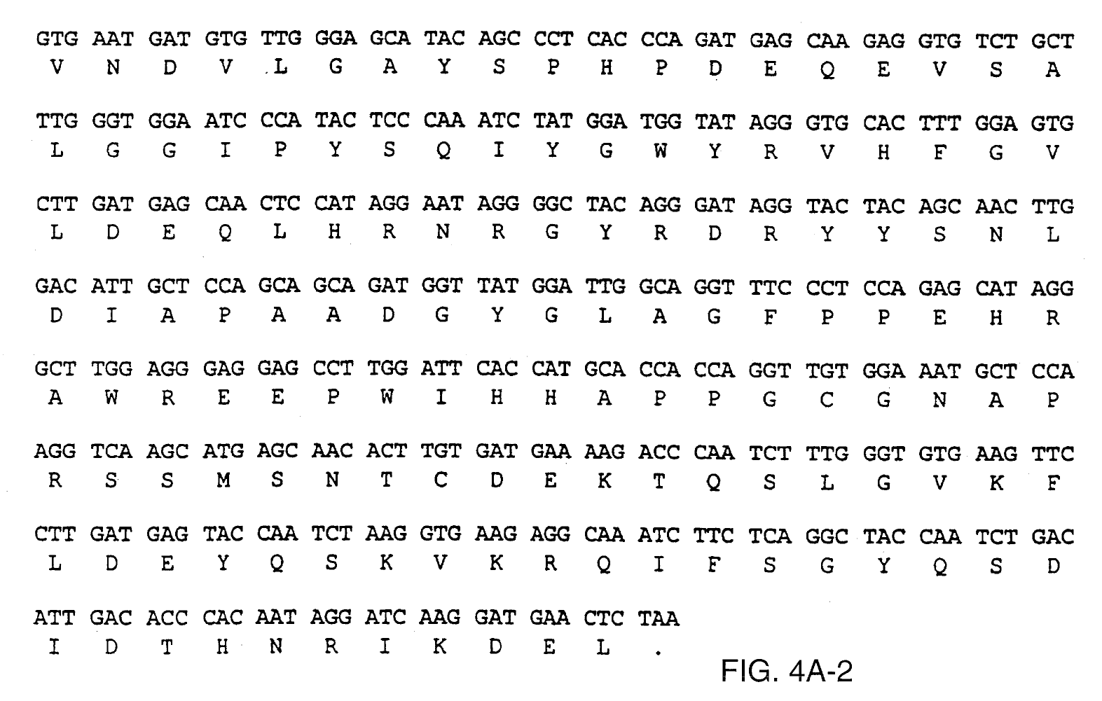

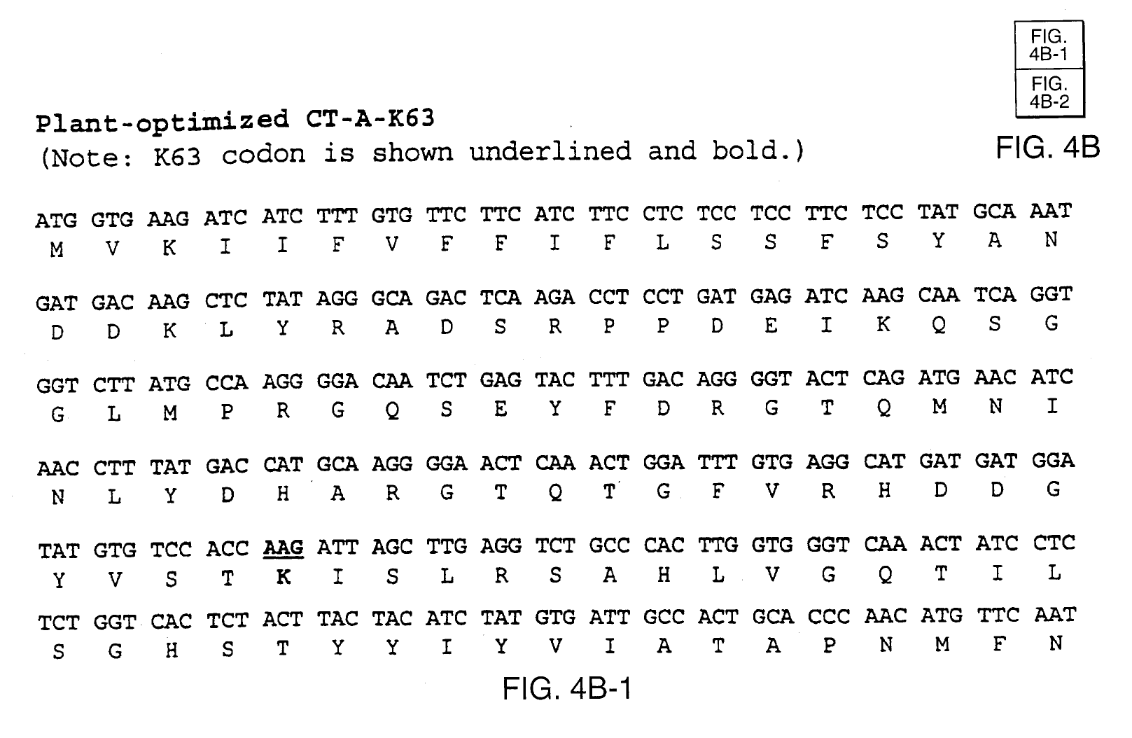

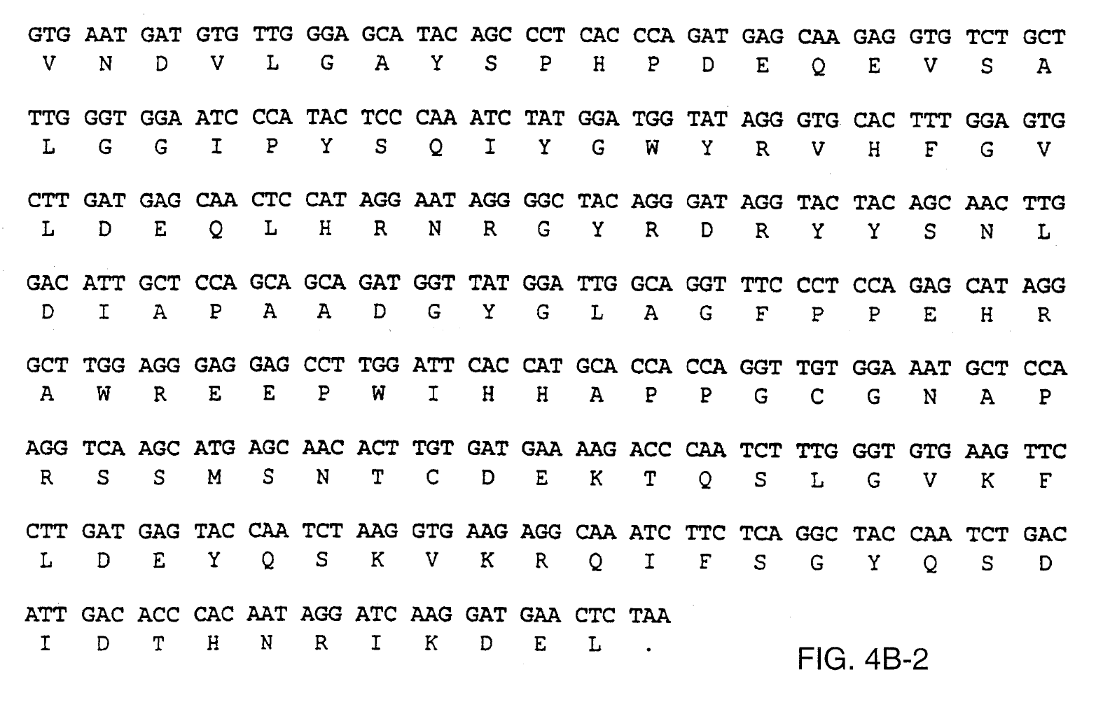

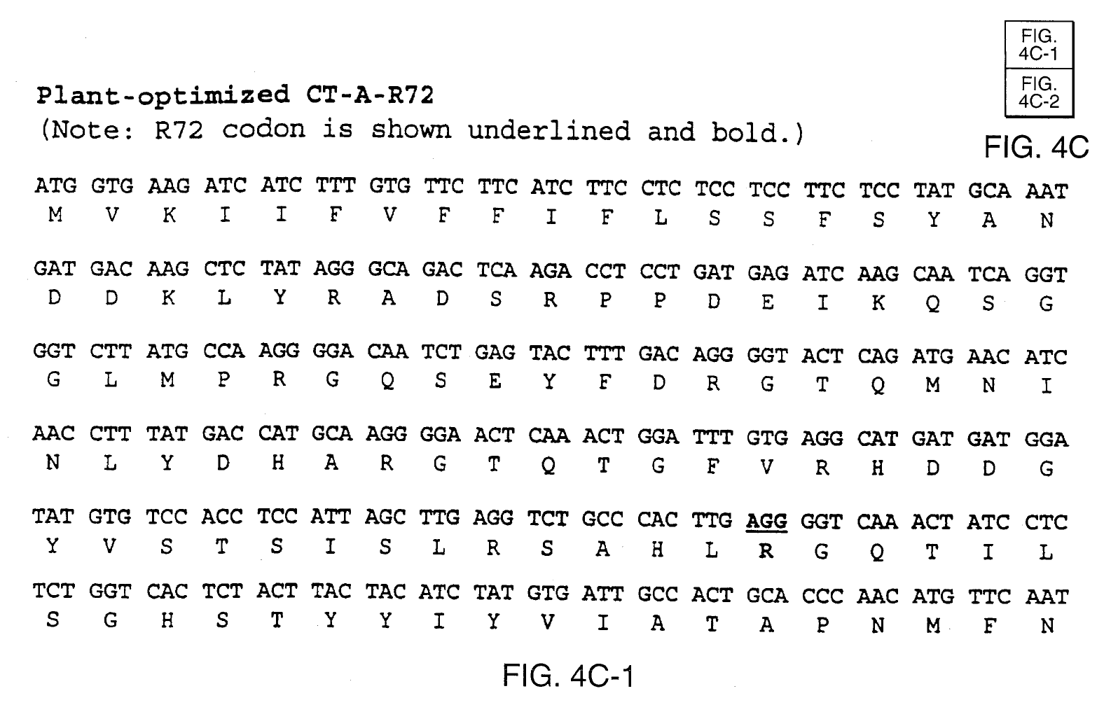

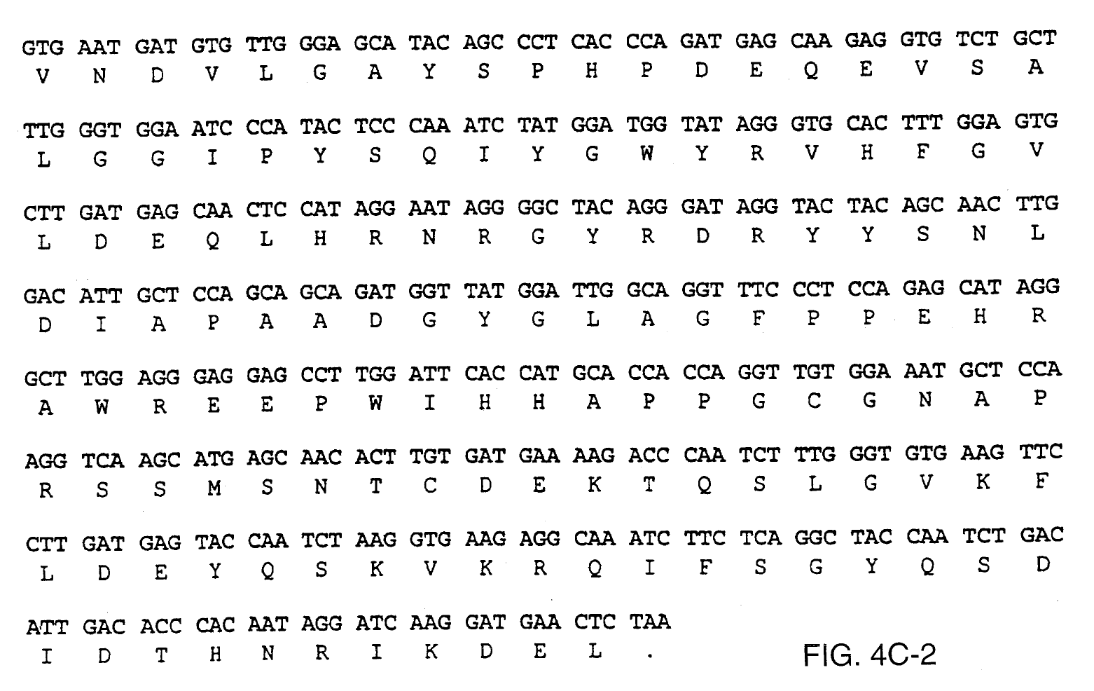

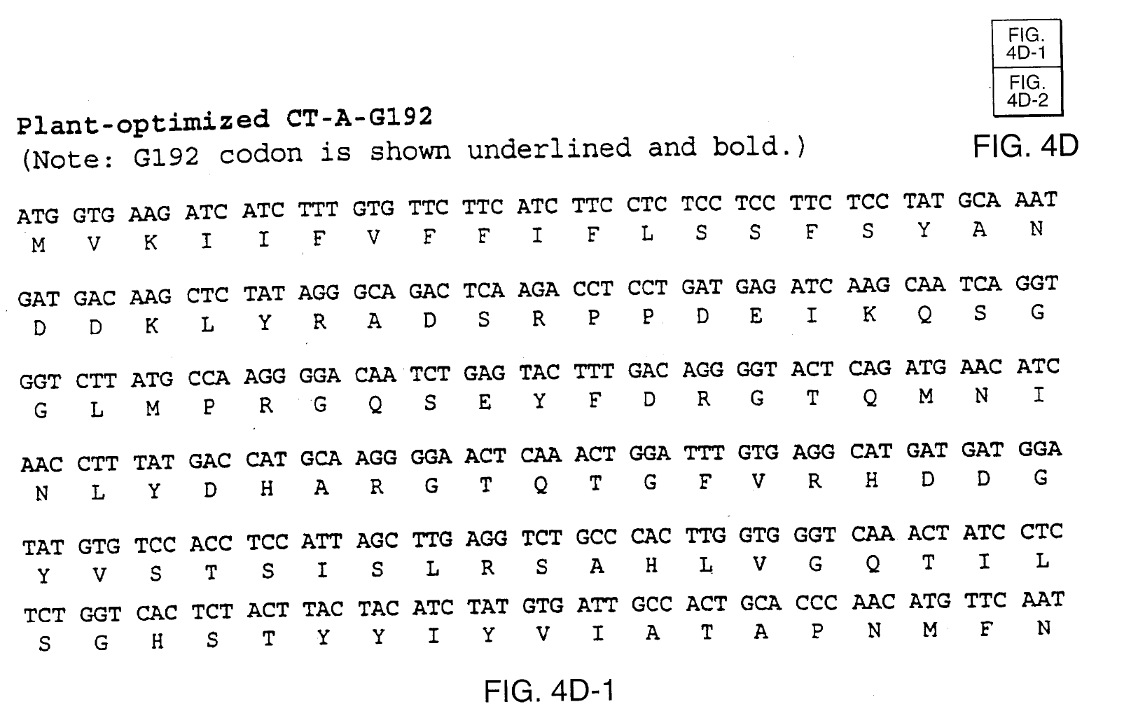

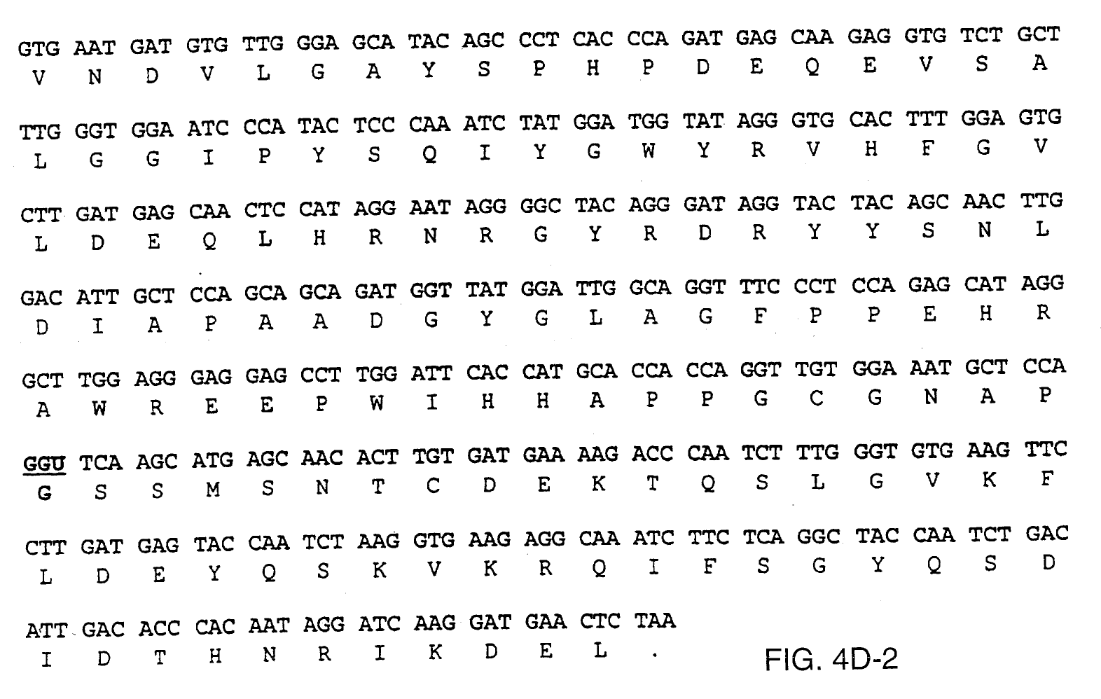

A CT-A or LT-A polypeptide of the invention preferably comprises a mutation at amino acid 61, 63, 72, 106, or 192; however, any mutation that reduces or eliminates enzymatic activity, and more particularly ADP ribosylating activity, of a CT-A or LT-A polypeptide is contemplated by the invention. Further, any mutation that prevents the cleavage of an A subunit into its fragments is contemplated by the invention. A polypeptide of the invention can comprise a single, double or multiple mutation (addition, deletion, or substitution of an amino acid or truncations of an amino acid) as compared to a wild-type LT-A sequence. For example, FIG. 4B shows a plant-optimized CT-A-K63 mutant nucleotide sequence and encoded amino acid sequence (SEQ ID NO:43). FIG. 4C shows a plant-optimized CT-A-R72 mutant nucleotide sequence and encoded amino acid sequence (SEQ ID NO:44). FIG. 4D shows a plant-optimized CT-A-G192 mutant nucleotide sequence and encoded amino acid sequence (SEQ ID NO:45).

Various strains and isolates of E. coli and V. cholerae occur and LT or CT polypeptides of any of these strains and isolates can be used in the present invention. LT-A and CT-A polypeptides of the invention can either be full-length polypeptides, fragments of polypeptides, or truncated segments of LT-A or CT-A polypeptides. For example fragments of LT-A or CT-A polypeptides can comprise 6, 10, 25, 50, 75, 100, 150, 200, 250, 300, or 350 amino acids of LT-A or CT-A polypeptides.

An LT-A or CT-A polypeptide of the invention can be combined or synthesized with a truncated LT-B or CT-B polypeptide, a fragment of an LT-B or CT-B polypeptide, or a full-length LT-B or CT-B polypeptide. For example a fragment of LT-B or CT-B polypeptide can comprise 6, 10, 25, 50, 75, 100, 125 or 150 amino acids of an LT-B or CT-B polypeptide. The LT-A and LT-B or CT-B may be from the same or different bacterial strains. The CT-A and CT-B or LT-B may be from the same or different bacterial strains. Further, one or more LT-A, LT-B, CT-A, and CT-B (i.e., 2, 3, 4, 5, 10, 25, or 50) polypeptides of the invention, from the same or different bacterial strains can be combined.

The polypeptides of the invention comprise at least one epitope that is recognized by an anti-LT or anti-CT antibody. Epitopes within the polypeptides can be identified by several methods. For example, a polypeptide of the invention can be isolated by methods such as immunoaffinity purification using a monoclonal antibody for the polypeptide. The isolated polypeptide sequence can then be screened. A series of short peptides, which together span the entire polypeptide sequence, can be prepared by proteolytic cleavage. By starting with, for example, 50-mer polypeptide fragments, each fragment can be tested for the presence of epitopes recognized in an anti-LT or anti-CT enzyme-linked immunosorbent assay (ELISA). Progressively smaller and overlapping fragments can then be tested from an identified 50-mer to map the epitope of interest.

Preferably, a polypeptide of the invention is produced recombinantly. A polynucleotide encoding a polypeptide of the invention can be introduced into an expression vector which can be expressed in a suitable expression system using techniques well known in the art. A variety of bacterial, yeast, plant, mammialian, and insect expression systems are available in the art and any such expression system can be used. A polypeptide of the invention can be isolated and purified from for example, a eukaryotic cell, such as a plant cell, by methods well known in the art such as immunoaffinity purification. Optionally, a polynucleotide encoding a polypeptide of the invention can be translated in a cell-free translation system.

If desired, a polypeptide of the invention can be produced as a fusion protein, which can also contain other amino acid sequences, such as amino acid linkers or signal sequences, as well as ligands useful in protein purification, such as glutathione-S-transferase, histidine tag, and staphylococcal protein A. Optionally, one or more antigens such as LT-B, CT-B, colonization antigens, virulence antigens, and epitopes thereof, and other compositions useful for the stimulation of a secretory immune response in an animal or human can be present in a fusion protein. More than one CT or LT polypeptide can be present in a fusion protein. If desired, various combinations of LT or CT polypeptides from different LT or CT strains or isolates can be included in a fusion protein.

LT and CT Polynucleotides

Polynucleotides of the invention contain less than an entire bacterial genome and can be single- or double-stranded DNA. Preferably, the polynucleotides are purified free of other components, such as proteins. The polynucleotides encode the CT and LT polypeptides described above. Polynucleotides of the invention can be isolated from a genomic library derived from nucleic acid sequences present in, for example, E. coli or V. cholerae cell cultures. An amplification method such as PCR can be used to amplify polynucleotides from either bacterial genomic RNA or cDNA encoding the polypeptides. The polynucleotides can also be synthesized in the laboratory, for example, using an automatic synthesizer.

The polynucleotides can comprise coding sequences for naturally occurring CT and LT polypeptides or can encode “altered” CT and LT sequences, i.e., which do not occur in nature. If desired, the polynucleotides can be cloned into an expression vector and transformed into, for example, bacterial, yeast, insect, plant, or mammalian cells so that the polypeptides of the invention can be expressed in and isolated from cell culture. The polynucleotides can be contained within a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, and cytomegalovirus and retroviral vectors, such as murine sarcoma virus, mouse mammary tumor virus, Moloney murine leukemia virus, and Rous sarcoma virus. Bacterial vectors, such as Salmonella ssp., Yersinia enterocolitica, Shigella spp., Vibrio cholerae, Mycobacterium strain BCG, Listeria monocytogenes, and Agrobacterium spp. can be used. Minichromosomes such as MC and MC1, bacteriophages, virus particles, virus-like particles, cosmids (plasmids into which phage lambda cos sites have been inserted) and replicons (genetic elements that are capable of replication under their own control in a cell) can also be used.

A mutation or mutations in LT or CT polynucleotides can be made by site-directed mutagenesis using conventional techniques. A library of mutant polynucleotides comprising single, double, or higher mutations, can also be prepared using random mutagenesis techniques. Mutagenesis techniques are described generally, e.g., in Current Protocols in Molecular Biology, Ausubel, F. et al. eds., John Wiley (1998), and random mutagenesis (also referred to as “DNA shuffling”) is the subject of U.S. Pat. Nos. 5,605,793, 5,811,238, 5,830,721, 5,834,252, and 5,837,458 to Stemmer et al. A polynucleotide comprising mutations of LT-A or CT-A can also be synthesized in a laboratory.

Preferably, a CT or LT polynucleotide of the invention is engineered such that the bacterial codons are systematically replaced by plant-preferred codons. For example, the coding sequence of LT or CT, or a portion thereof, can be analyzed for its codon usage. This can then be compared with the frequency of codon usage in abundant proteins found in a particular plant. See, e.g. WO 96/12801. The codons of the polynucleotide which have low or zero frequency of use in a plant can be modified by, for example, site directed mutagenesis or the polynucleotide can be synthesized in the laboratory. The codon modifications are “altered” made to conform with the plant codons used in the genes for the abundantly expressed plant proteins.

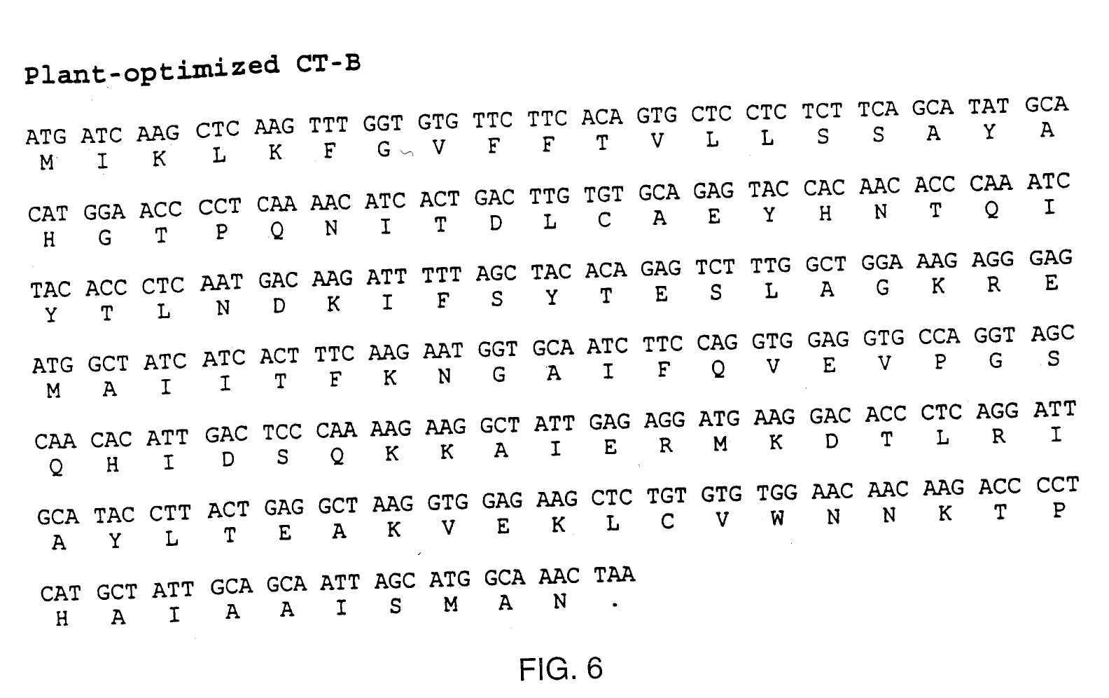

Thus a polymercleotide wherein at least one codon is “altered” to a plant-preferred codon is simply a polyonucleotide having a sequence that is different from the wild type sequence in that it contains at least one codon that is used preferentially in plants. Mutant polynucleotide sequences according to the invention are preferably at least 50% homologous, preferably 60%, 70%, 80% and 90% homologous with the wild type sequence from which the mutant sequence was derived. Further, segments of codons with possible poly-A signal sequences can be modified to other codons for the same amino acids. Further, cryptic signal sequences and potential methylation sites can be modified. See Example 1 and WO 96/12801. See also, FIG. 4B shows a plant-optimized CT-A-K63 mutant nucleotide sequence and encoded amino acid sequence; FIG. 4C shows a plant-optimized CT-A-R72 mutant nucleotide sequence and encoded amino acid sequence; FIG. 4D shows a plant-optimized CT-A-G192 mutant nucleotide sequence and encoded amino acid sequence; FIG. 6 shows a nucleotide sequence encoding CT-B, which has been optimized for expression in plants. The replacement or substitution of plant-preferred codons for the corresponding bacteria-preferred codon enhances the expression of the CT and/or LT polynucleotides and can facilitate expression of the encoded polypeptide or polypeptides in a particular part, e.g., the fruit or tuber, of the plant. A CT or LT polynucleotide sequence that has had at least 1, 2, 3, 4, 5, 10, 20, 50, or more codons modified to plant-preferred codons is said to be plant-optimized. Preferably, plant-optimization further comprises modification of codons encoding possible signal sequences, intron splice sites, and methylation sites.

Expression Vectors

If desired, the LT and CT polynucleotides of the invention can be cloned into an expression vector and transformed into, for example, bacterial, yeast, insect, plant, or mammalian cells so that the polypeptides of the invention can be expressed in and isolated from cell culture or plants. The polynucleotides can be contained within a plasmid, a virus, a bacterial vector, minichromosomes such as MC and MC1, bacteriophages, virus particles, virus-like particles, cosmids (plasmids into which phage lambda cos sites have been inserted) and replicons (genetic elements that are capable of replication under their own control in a cell), as described above.

Preferably, an expression vector comprising a polynucleotide of the invention has an LT or CT encoding sequence operably linked to a plant functional promoter. Preferred plant promoters include CaMV 35S, patatin, mas, and granule-bound starch synthase promoters. Other promoters, and enhancers that can be employed are listed below. A particularly preferred promoter is the CaMV 35S promoter with dual enhancer. A preferred polynucleotide comprises a tobacco mosaic virus (TMV) 5′ untranslated region (UTR) (omega) or fragments thereof between the promoter and a polynucleotide sequence. A TMV 5′ UTR facilitates translation of the coding sequence. A polynucleotide may further comprise a TMV 3′-UTR or fragments thereof, which can further facilitate translation. Zeyenko et al., FEBS Lett. 354:271-273 (1994); Leathers et al., Mol. Cell. Biol. 13:5331-5347 (1993); Gallie et al., Nucl. Acids Res. 20:4631-4638.

An LT-A or CT-A polynucleotide of the invention can be operably linked to a plant functional promoter, and can optionally be operably linked to a polynucleotide encoding LT-B or CT-B in an expression vector. The polynucleotide encoding the LT-B or CT-B subunit may be operably linked to its own promoter, independent of the promoter for the A subunit. In a preferred embodiment of the invention, a polynucleotide has a tobacco etch virus (TEV) 5′ untranslated region provided between the promoter for the LT-B or CT-B polynucleotide and the LT-B or CT-B polynucleotide sequence to enhance translation efficiency. Optionally, the polynucleotide can further comprise a TEV poly (A) tail. Gallie et al., Gene. 165:233-238 (1995); Carrington et al., J. Virol. 64:1590-1597 (1990). Also, a polynucleotide of the invention can encode a microsomal retention signal sequence, such as SEKDEL (SEQ ID NO:59), in order to increase retention of the expressed polypeptide in the cell, e.g., further to assembly of holotoxin. See WO96/12801.

Some exemplary plant functional promoters, which can be used to express a structural gene of the present invention, are among the following: U.S. Pat. No. 5,352,605 and U.S. Pat. No. 5,530,196—CaMV 35S and 19S promoters; U.S. Pat. No. 5,436,393—patatin promoter; U.S. Pat. No. 5,436,393—B33 promoter sequence of a patatin gene derived from Solanum tubersum, and which leads to a tuber specific expression of sequences fused to the B33 promoter; WO 94/24298—tomato E8 promoter; U.S. Pat. No. 5,556,653—tomato fruit promoters; U.S. Pat. Nos. 5,614,399 and 5,510,474—plant ubiquitin promoter system; U.S. Pat. No. 5,824,865—5′ cis-regulatory elements of abscisic acid-responsive gene expression; U.S. Pat. No. 5,824,857—promoter from badnavirus, rice tungro bacillifonn virus (RTBV); U.S. Pat. No. 5,789,214—chemically inducible promoter fragment from the 5′ flanking region adjacent the coding region of a tobacco PR-1a gene; U.S. Pat. No. 5,783,394—raspberry drul promoter; WO 98/31812 strawberry promoters and genes; U.S. Pat. No. 5,773,697—napin promoter, phaseolin promoter, and DC3 promoter; U.S. Pat. No. 5,723,765—LEA promoter; U.S. Pat. No. 5,723,75 7—5′ transcriptional regulatory region for sink organ specific expression; U.S. Pat. No. 5,723,751—G-box related sequence motifs, specifically 1 wt and PA motifs, which function as cis-elements of promoters, to regulate the expression of heterologous genes in transgenic plants; U.S. Pat. No. 5,633,440—P 119 promoters and their use; U.S. Pat. No. 5,608,144—Group 2 (Gp2) plant promoter sequences; U.S. Pat. No. 5,608,143—nucleic acid promoter fragments derived from several genes from corn, petunia and tobacco; U.S. Pat. No. 5,391,725—promoter sequences from the nuclear gene for chloroplast GS2 glutamine synthetase and from two nuclear genes for cytosolic GS3 glutamine synthetase in the pea plant, Pisum sativum; U.S. Pat. No. 5,378,619—full-length transcript promoter from flagwort mosaic virus (FMV); U.S. Pat. No. 5,689,040—isocitrate lyase promoter; U.S. Pat. No. 5,633,438—microspore-specific regulatory element; U.S. Pat. No. 5,595,896—expression of heterologous genes in transgenic plants and plant cells using plant asparagine synthetase promoters; U.S. Pat. No. 4,771,002—promoter region that drives expression of a 1450 base TR transcript in octopine-type crown gall tumors; U.S. Pat. No. 4,962,028—promoter sequences from the gene from the small subunit of ribulose-1,5-bisphosphate carboxylase; U.S. Pat. No. 5,491,288—Arabidopsis histone H4 promoter; U.S. Pat. No. 5,767,363—seed-specific plant promoter; U.S. Pat. No. 5,023,179—21 bp promoter element which is capable of imparting root expression capability to a rbcS-3A promoter, normally a green tissue specific promoter; U.S. Pat. No. 5,792,925—promoters of tissue-preferential transcription of associated DNA sequences in plants, particularly in the roots; U.S. Pat. No. 5,689,053—Brassica sp. polygalacturonase promoter; U.S. Pat. No. 5,824,863—seed coat-specific cryptic promoter region; U.S. Pat. No. 5,689,044—chemically inducible nucleic acid promoter fragment isolated from the tobacco PR-1a gene is inducible by application of a benzo-1,2,3-thiadiazole, an isonicotinic acid compound, or a salicylic acid compound; U.S. Pat. No. 5,654,414—promoter fragment isolated from a cucumber chitinase/lysozyme gene that is inducible by application of benzo-1,2,3-thiadiazole; U.S. Pat. No. 5,824,872—constitutive promoter from tobacco that directs expression in at least ovary, flower, immature embryo, mature embryo, seed, stem, leaf and root tissues; U.S. Pat. No. 5,223,419—alteration of gene expression in plants; U.S. Pat. No. 5,290,924—recombinant promoter for gene expression in mo-mocotyledenous plants; WO 95/21248—method for using TMV to overproduce peptides and proteins; WO 98/05199—nucleic acid comprising shoot meristem-specific promoter and regulated sequence; EP-B-0122791—phaseolin promoter-and structural gene; U.S. Pat. No. 5,097,025—plant promoters (sub domain of CaMV 35S); WO 94/24294—use of tomato E8-derived promoters to express heterologous genes, e.g. 5-adenosylmethionine hydrolase in ripening fruit; U.S. Pat. No. 5,801,027—method of using transactivation proteins to control gene expression in transgenic plants; U.S. Pat. No. 5,821,398—DNA molecules encoding inducible plant promoters and tomato Adh2 enzyme; WO 97/47756—synthetic plant core promoter and upstream regulatory element; U.S. Pat. No. 5,684,239—monocot having dicot wound inducible promoter; U.S. Pat. No. 5,110,732—selective gene expression in plants; U.S. Pat. No. 5,106,739—CaMV 35S enhanced mannopine synthase promoter and method for using the same; U.S. Pat. No. 5,420,034—seed specific transcription regulation; U.S. Pat. No. 5,623,067—seed specific promoter region; U.S. Pat. No. 5,139,954—DNA promoter fragments from wheat; WO 95/14098—chimeric regulatory regions and gene cassettes for use in plants; WO 90/13658—production of gene products to high levels; U.S. Pat. No. 5,670,349—HMG promoter expression system and post harvest production of gene products in plants and plant cell cultures; U.S. Pat. No. 5,712,112—gene expression system comprising the promoter region of the alpha amylase genes in plants.

Preferably, an expression vector comprises one or more enhancers. Some enhancers that can be used with the present invention are among the following: U.S. Pat. No. 5,424,200 and U.S. Pat. No. 5,196,525—CAMV 35S enhancer sequences; U.S. Pat. No. 5,359,142, U.S. Pat. No. 5,322,938, U.S. Pat. No. 5,164,316, and U.S. Pat. No. 5,424,200—tandemly duplicated CaMV 35S enhancers; WO 87/07664 —ω region of TNW; WO 98/14604—intron 1 and/or intron 2 of the PAT1 gene.; U.S. Pat. No. 5,593,874—HSP70 introns that when present in a non-translated leader of a chimeric gene enhance expression in plants; U.S. Pat. No. 5,710,267, U.S Pat. No. 5,573,932, and U.S. Pat. No. 5,837,849—plant enhancer element capable of being bound by an OCS transcription factor; U.S. Pat. No. 5,290,924—a maize Adh1 intron; JP 8256777—translation enhancer sequence.

An expression vector of the present invention can also include a transcription termination sequence functional in a plant host. Exemplary termination sequences include nopaline synthase (nos) (B3evan, Nucleic Acids Res.,: 12: 8711-8721(1984)), vegetative storage protein (vsp) (Mason et al., Plant Cell. 5:241-251 (1993)) and proteinase inhibitory (pin2) (An et al. 1989) termination sequences.

Preferably, an expression vector comprises a selectable marker in addition to a polynucleotide of the invention. Examples of selectable markers include a kanamycin gene, a β-glucuronidase gene, a neomycin transferase gene, a tfdA gene, a Pat gene, and a bar gene. An expression vector can also comprise an E. coli origin of replication, for example the ColE1 or pBR322 origin of replication, to facilitate replication of the vector in E. coli. An expression vector of the invention can also comprise an A. tumefaciens origin of replication, to permit replication of the vector therein, such as when A. tumefaciens is to be used for plant transformation.

Expression vectors of the invention can be designed to comprise promoters that direct polypeptide expression in particular parts of the plant. For example, expression vectors including the CaMV 35S promoter and a polynucleotide of the invention can be used to constitutively transform plants so that a polypeptide expressed by a polynucleotide of the invention is produced in the leaves of the plant. This allows for rapid analysis of polynucleotide expression and biochemical characterization of polynucleotide products.

Expression vectors comprising a 2S albumin promoter and a polynucleotide of the invention can be used to cause seed-specific polynucleotide expression to create the production of a polypeptide of the invention in seed tissues, for example canola (Brassica napus) seeds.

Expression vectors comprising a patatin promoter or a soybean vspB promoter and a polynucleotide of the invention can be used to cause tuber-specific polynucleotide expression and tuber specific production of a polypeptide in tuber tissues such as potato (Solanum tubersum).

Expression vectors comprising fruit ripening specific promoters and polynucleotides of the invention can be used to transform plants that produce a polypeptide of the invention in ripened fruit, such as banana (Musa acuminata).

Expression vectors optimized for the expression of a CT or LT holotoxin can also be constructed. For example, the A and B subunits of LT and CT are combined in the ratio of 1:5. Therefore, the transcription levels for these polynucleotides encoding LT and CT in transgenic plants can be modulated to approximate a similar ratio of mRNAs for the A and B subunits in the transformed cells. This can be accomplished by using promoters in the expression vector having different activities for the A and B subunits. See WO 96/12801.

Transformation and Regeneration of Plants with Polynucleotides and Expression Vectors

A further aspect of the present invention is a eukaryotic or prokaryotic cell that comprises, e.g., is transformed with, a polynucleotide of the invention. Preferably, the cell is a plant cell, but other types of cells, such as insect, mammalian, and bacterial cells are contemplated. Whenever a plant cell is employed, it is preferred that the polynucleotide is integrated into the nuclear genome of the plant cell to ensure its stability and passage into the germline. A polynucleotide of the invention can also in some cases be maintained outside the chromosome, such as in the mitochondrion, chloroplast or cytoplasm. A preferred mode of transfer of a polynucleotide to an insect cell is via viral transport, where replication can be maintained extrachromosomally or by integration. Methods of transfer of polynucleotides into mammalian and bacterial cells are well known in the art.

A transformed plant cell is preferably one from a plant that can be consumed as a foodstuff or that expresses the desired protein or polypeptide in a readily isolateable form. Representative plants include tobacco, banana, tomato, potato, carrot, soybean, corn, rice, wheat, and sunflower. A transgenic plant seed transformed with a polynucleotide of the invention, which is obtained by propagation of a transgenic plant, is yet a further aspect of the invention.

Among the principal methods for effecting transfer of foreign nucleic acid constructs into plants is the A. tumefaciens transformation technique. This method is based upon the etiologic agent of crown gall, which afflicts a wide range of dicotyledons and gymnosperms. Where the target plant host is susceptible to infection, the A. tumefaciens system provides high rates of transformation and predictable chromosome integration patterns.

Agrobacterium, which normally infects a plant at wound sites, carries a large extrachromosomal element called Ti (tumor inducing) plasmid. Ti plasmids contain two regions required for tumor induction. One region is the T-DNA (transferred DNA), which is the DNA sequence that is ultimately stably transferred to plant genomic DNA. The other region is the vir (virulence) region, which has been implicated in the transfer mechanism. Although the vir region is required for stable transformation, the vir region DNA is not transferred to the infected plant. Transformation of plant cells mediated by infection with Agrobacterium and subsequent transfer of the T-DNA have been well documented. Bevan et al., Int. Rev. Genet. 16:357 (1982). The Agrobacterium system is well developed and permits routine transformation of DNA into the plant genome of a variety of plant tissues. For example, tobacco, tomato, sunflower, cotton, rapeseed, potato, poplar, and soybean can be transformed with the Agrobacterium system.

Preferably, where A. tumefaciens-mediated transformation of plants with a polynucleotide of the invention is used, flanking T-DNA border regions of A. tumefaciens are provided. T-DNA border regions are 23-25 base pair direct repeats involved in the transfer of T-DNA to the plant genome. The flanking T-DNA border regions bracket the T-DNA and signal the polynucleotide that is to be transferred and integrated into the plant genome. Preferably, a polynucleotide or expression vector of the invention comprises at least one T-DNA border, particularly the right T-DNA border. Optionally, a polynucleotide to be delivered to a plant genome is sandwiched between the left and right T-DNA borders. The borders may be joined to an expression vector or polynucleotide by any conventional means obtained from any Ti or Ri (see below) plasmid and may be joined to an expression vector or polynucleotide by any conventional means.

Typically, a vector containing the polynucleotide to be transferred is first constructed and replicated in E. coli. This vector contains at least one right T-DNA border region, and preferably a left and a right border region flanking the desired polynucleotide. A selectable marker (such as a gene encoding resistance to an antibiotic such as kanamycin) can also be present to permit ready selection of transformed cells. The E. coli vector is next transferred to Agrobacterium, which can be accomplished via a conjugation mating system or by direct uptake. Once inside the Agrobacterium, the vector containing the polynucleotide can undergo homologous recombination with a Ti plasmid of the Agrobacterium to incorporate the T-DNA into a Ti plasmid. A Ti plasmid contains a set of inducible vir genes that effect transfer of the T-DNA to plant cells.

Alternatively, the vector comprising the polynucleotide can be subjected in trans to the vir genes of the Ti plasmids. In a preferred aspect, a Ti plasmid of a given strain is “disarmed,” whereby the one genes of the T-DNA is eliminated or suppressed to avoid formation of tumors in the transformed plant, but the vir genes provided in trans still effect transfer of T-DNA to the plant host. See, e.g., Hood, Transgenic Res. 2: 208-218 (1993); Simpson, Plant Mol. Biol. 6: 403-415 (1986). For example, in a binary vector system, an E. coli plasmid vector is constructed comprising a polynucleotide of interest flanked by T-DNA border regions and a selectable marker. The plasmid vector is transformed into E. coli and the transformed E. coli is then mated to Agrobacterium by conjugation. The recipient Agrobacterium contains a second Ti plasmid (helper Ti plasmid) that contains vir genes, but has been modified by removal of its T-DNA fragment. The helper Ti plasmid will supply proteins necessary for plant cell infection, but only the E. coli modified T-DNA plasmid will be transferred to the plant cell.

The A. tumefaciens system permits routine transformation of a variety of plant tissues. See, e.g., Chilton, Scientific American 248:50 (1983); Gelvin, Plant Physiol. 92: 281-285 (1990); Hooykaas, Plant Mol Biol. 13: 327-336 (1992); Rogers et al., Science 227: 1229-1231 (1985). Representative plants that have been transformed with this system and representative references are listed in Table 1. Other plants having edible parts, or which can be processed to afford isolated protein, can be transformed by the same methods or routine modifications thereof.

TABLE I

| Plant | Reference |
|---|---|
| Tobacco | Barton, K. et al., (1983) Cell 32, 1033 |
| Tomato | Fillatti, J. et al., (1987) Bio/Technology 5, 726-730 |
| Potato | Hoekema, A. et al., (1989) Bio/Technology 7:273-278 |
| Eggplant | Filipponee, E. et al., (1989) Plant Cell Rep. 8:370-373 |
| Pepino | Atkinson, R. et al., (1991) Plant Cell Rep. 10:208-212 |
| Yam | Shafer, W. et al., (1987) Nature. 327:529-532 |
| Soybean | Deizer, B., et al., (1990) Crop Sci. 30:320-322 |
| Pea | Hobbs, S. et al., (1989) Plant Cell Rep. 8:274-277 |
| Sugar beet | Kallerhoff, J. et al., (1990) Plant Cell Rep. 9:224-228 |
| Lettuce | Michelmore, R., et al., (1987) Plant Cell Rep. 6:439-442 |
| Bell pepper | Liu, W. et al., (1990) Plant Cell Rep. 9:360-364 |
| Celery | Liu, C-N. et al., (1992) Plant Mol. Biol. 1071-1087 |
| Carrot | Liu, C-N. et al., (1992) Plant Mol. Biol. 1071-1087 |
| Asparagus | Deibriel, B. et al., (1993) Plant Cell Rep. 12:129-132 |
| Onion | Dommisse, E. et al., (1990) Plant Sci. 69:249-257 |
| Grapevine | Baribault, T., et al., (1989) Plant Cell Rep. 8:137-140 |
| Muskmelon | Fang, G., et al., (1990) Plant Cell Rep. 9:160-164 |
| Strawberry | Nehra, N. et al., (1990) Plant Cell Rep. 9:10-13 |
| Rice | Raineri, D. et al., (1990) Bio/Technology. 8:33-38 |
| Sunflower | Schrammeijer, B. et al., (1990) Plant Cell Rep. 9:55-60 |
| Rapeseed/ | Pua, E. et al., (1987) Bio/Technology 5. 815 |
| Canola | |
| Wheat | Mooney, P. et al., (1991) Plant Cell Tiss. Organ Cult. |
| | 25:209-218 |
| Oats | Donson, J. et al., (1988) Virology. 162:248-250 |
| Maize | Gould, J. et al., (1991) Plant Physiol. 95:426-434 |
| Alfalfa | Chabaud, M. et al., (1988) Plant Cell Rep. 7:512-516 |
| Cotton | Umbeck, P. et al., (1987) Bio/Technology. 5:263-266 |
| Walnut | McGranahan, G. et al., (1990) Plant Cell Rep. 8:512-516 |
| Spruce/ | Ellis, D. et al., (1989) Plant Cell Rep. 8:16-20 |
| Conifer | |
| Poplar | Pythoud, F. et al., (1987) Bio/Technology 5:1323 |
| Apple | James, D. et al., (1989) Plant Cell Rep. 7:658-661 |

Other Agrobacterium strains such as A. rhizogenes can be used as a vector for plant transformation. A. rhizogenes, which incites root hair formation in many dicotyledonous plant species, carries a large extra-chromosomal element called a Ri (root-including) plasmid, which functions in a manner analogous to the Ti plasmid of A. tumefaciens. Transformation using A. rhizogenes has developed analogously to that of A. tumefaciens and has been used successfully, e.g., to transform alfalfa and poplar. Sukhapinda et al., Plant Mol. Biol. 8:209 (1987).

Methods of inoculation of the plant tissue vary depending upon the plant species and the Agrobacterium delivery system. A convenient approach is the leaf disc procedure which can be performed with any tissue explant that provides a good source for initiation of whole plant differentiation. The addition of nurse tissue may be desirable under certain conditions. Other procedures such as in vitro transformation of regenerating protoplasts with A. tumefaciens may be followed to obtain transformed plant cells as well.

Direct gene transfer procedures can be used to transform plants and plant tissues without the use of Agrobacterium plasmids. Potrykus, Bio/Technology. 8:535-542 (1990); Smith et al. Crop Sci., 35: 01-309 (1995). Direct transformation involves the uptake of exogenous genetic material into plant cells or protoplasts. Such uptake can be enhanced by use of chemical agents or electric fields. For example, a polynucleotide of the invention can be transformed into protoplasts of a plant by treatment of the protoplasts with an electric pulse in the presence of the protoplast using electroporation. For electroporation, the protoplasts are isolated and suspended in a mannitol solution. Supercoiled or circular plasmid DNA comprising a polyllucleotide of the invention is added. The solution is mixed and subjected to a pulse of about 400 V/cm at room temperature for about 10 to 100 microseconds. A reversible physical breakdown of the membrane occurs such that the foreign genetic material is transferred into the protoplasts. The foreign genetic material can then be integrated into the nuclear genome. Several monocot protoplasts have also been transformed by this procedure including rice and maize.

Liposome fusion is also an effective method for transformation of plant cells. In this method, protoplasts are brought together with liposomes carrying a polynucleotide of the invention. As the membranes merge, the foreign gene is transferred to the protoplasts. Dehayes et al., EMBO J. 4:2731 (1985). Similarly, direct gene transfer using polyethylene glycol (PEG) mediated transformation has been carried out in N. tabacum (a dicot) and Lolium multiflorum (a monocot). Direct gene transfer is effected by the synergistic interaction between Mg⁺², PEG, and possibly Ca⁺². Negrutiu et al., Plant Mol. Biol. 8: 363 (1987). Alternatively, exogenous DNA can be introduced into cells or protoplasts by microinjection of a solution of plasmid DNA comprising a polynucleotide of the invention directly into the cell with a finely pulled glass needle.

Direct gene transfer can also be accomplished by particle bombardment (or microparticle acceleration), which involves bombardment of plant cells by microprojectiles carrying a polynucleotide of the invention. Klein et al., Nature 327:70 (1987); Sanford, Physiol. Plant. 79: 206-209 (1990). In this procedure, chemically inert metal particles, such as tungsten or gold, are coated with a polynucleotide of the invention and accelerated toward the target plant cells. The particles penetrate the cells, carrying with them the coated polynucleotide. Microparticle acceleration has been shown to lead to both transient expression and stable expression in cells suspended in cultures, protoplasts, and immature embryos of plants, including onion, maize, soybean, and tobacco. McCabe et al., Bio/Technology. 6: 923 (1988).

Additionally, DNA viruses can be used as gene vectors in plants. For example, a cauliflower mosaic virus carrying a modified bacterial methotrexate-resistance gene has been used to infect a plant. The foreign gene systematically spreads throughout the plant. Brisson et al., Nature 310:511 (1984). The advantages of this system are the ease of infection, systemic spread within the plant, and multiple copies of the gene per cell.

Once plant cells have been transformed, there are a variety of methods for regenerating plants. The particular method of regeneration will depend on the starting plant tissue and the particular plant species to be regenerated. Many plants can be regenerated from callus tissue derived from plant explants, including, but not limited to corn, rice, barley, wheat, rye, sunflower, soybean, cotton, rapeseed, and tobacco. Regeneration of plants from tissue transformed with A. tumefaciens has been demonstrated in plants including, but not limited to sunflower, tomato, white clover, rapeseed, cotton, tobacco, potato, maize, rice, and numerous vegetable crops. Plant regeneration from protoplasts is a particularly useful technique and has been demonstrated in plants including, but not limited to tobacco, potato, poplar, corn, and soybean. Evans et al., Handbook of Plant Cell Culture 1, 124 (1983).

Plants Expressing CT and LT

The invention includes whole plants, plant cells, plant organs, plant tissues, plant seeds, protoplasts, callus, cell cultures, and any group of plant cells organized into structural and/or functional units capable of expressing at least a polynucleotide of the invention. Preferably, whole plants, plant cells, plant organs, plant tissues, plant seeds, protoplasts, callus, cell cultures, and any group of plant cells produce 0.001, 0.01, 1, 5, 10, 25, 50, 100, 500, or 1000 μg of polypeptide of the invention per gram of total soluble plant material. The invention further comprises LT-A, LT-B, CT-A, CT-B, LT holotoxin, and CT holotoxin polypeptides of the invention isolated, purified, or partially purified from the plant cells, or plant tissue in which they were produced.

Extracts of plant tissue can be assayed for ganglioside binding dependant detection of LT-A, LT-B, CT-A, CT-B, LT holotoxin, and CT holotoxin and mutants thereof by ELISA. Briefly, GM-X ganglioside (Sigma) dissolved in carbonate buffer can be coated on, for example polystyrene ELISA plates. After an approximately 1 hour incubation at room temperature the buffer is washed off with for example, PBS and the wells blocked for non-specific binding with 5% milk in PBS. Plant extract samples to be assayed are loaded into the wells. To obtain a standard curve for quantification of the LT or CT polypeptides different dilutions of bacterially derived LT or CT polypeptides can be loaded on the same plate. The plates are washed with buffer after 1 hour incubation at room temperature. Goat-antiserum or rabbit antiserum to LT-A, LT-B, CT-A, CT-B, LT holotoxin, or CT holotoxin or mutants thereof, are diluted in buffer and BSA and incubated in the well for 1 hour at room temperature. After washing 4 times with buffer, the wells are probed with, for example rabbit antiserum against goat IgG conjugated with alkaline phosphatase diluted in buffer and BSA. After washing with buffer the wells are incubated with nitrophenyl phosphate substrate in diethanolamime buffer. After incubation for 10-30 minutes the reaction is stopped by adding NaOH and the absorbance read at 410 nm. Holotoxin assembly can be demonstrated by using a detector antibody specific for an A subunit that does not react with a B subunit. Because an A subunit does not bind to ganglioside unless assembled into holotoxin with B subunits, the ganglioside dependant ELISA for an A subunit demonstrates holotoxin assembly.

The toxic effect of the polypeptides of the invention on a plant in which the polypeptides are produced can be tested by comparing the growth of plants producing the polypeptides of the invention to plants that do not produce LT or CT polypeptides, and to plants that produce wild-type LT or CT. Preferably, plants producing the polypeptides of the invention have a same or similar rate of growth as plants that do not produce LT or CT polypeptides and have a greater rate of growth than plants that produce wild-type LT-A or CT-A.

Compositions Comprising LT and CT Polypeptides

The invention provides LT and CT polypeptide compositions in whole plants, plant cells, plant organs, plant seeds, protoplasts, callus, cell cultures, and any group of plant cells organized into structural and/or functional units capable of expressing at least a polynucleotide of the invention. The plant matter is preferably administered to a human or animal orally. The invention further comprises LT-A, LT-B, CT-A, CT-B, LT holotoxin, and CT holotoxin polypeptides of the invention that have been isolated, purified, or partially purified from the plant cells, or plant tissue in which they were produced. For example, in an extract or juice of a fruit of the plant. The invention also provides compositions comprising LT or CT polynucleotides.

Compositions of the invention can comprise a pharmaceutically acceptable carrier. The carrier should not itself induce the production of antibodies harmful to the host. Pharmaceutically acceptable carriers are, well known to those in the art. Such carriers include, but are not limited to, large, slowly metabolized, macromolecules, such as proteins, polysaccharides such as latex functionalized sepharose, agarose, cellulose, cellulose beads and the like, polylactic acids, polyglycolic acids, polymeric amino acids such as polyglutamic acid, polylysine, and the like, amino acid copolymers, peptoids, lipitoids, and inactive virus particles.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art. Compositions of the invention can also contain liquids or excipients, such as water, saline, glycerol, dextrose, malodextrin, ethanol, or the like, singly or in combination, as well as substances such as wetting agents, emulsifying agents, or pH buffering agents. Liposomes can also be used as a carrier for a composition of the invention.

If desired, co-stimulatory molecules, which improve immunogen presentation to lymphocytes, such as B7-1 or B7-2, or cytokines such as IL-2, and IL-12, can be included in a composition of the invention. Optionally, adjuvants can also be included in a composition. Adjuvants which can be used include, but are not limited to MF59-0, aluminum hydroxide, N-acetyl-muramyl-L-threonyl-D-isoglutamine (thr-MDP), N-acetyl-nor-muramnyl-L-alanyl-D-isoglutamine (CGP 11637), referred to as nor-MD P), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The compositions of the invention can comprise a sustained release formulation, enteric formulations, tablets, chewable tablets, capsules, solutions, parenteral solutions, intranasal sprays or powders, troches, suppositories, transdermal patches and suspensions. In general, compositions contain from about 0.01, 0.1, 1, 3, 5, 10, 20, 30, 40, 50, 60, 70, or 80% of the polypeptides or polynucleotides of the invention in total, depending on the desired doses and the type of composition to be used.

Method of Eliciting An Immune Response

LT or CT polypeptides of the invention can be used to elicit an immune response in animals such as cattle, swine, mice, rabbits, fowl, such as chickens, ducks, and geese, chimps, baboons, and macaques, and in humans. Preferably, LT or CT polypeptides elicit anti-LT or anti-CT IgG and/or IgA antibodies. Elicitation of anti-LT or anti-CT antibodies can be used, inter alia, to provide model systems to optimize anti-LT or anti-CT antibody responses to E. coli or V. cholerae and to provide prophylactic or therapeutic treatment against E. coli or V. cholerae.

Detection and/or quantification of anti-LT or anti-CT antibody titers after delivery of an LT or CT polypeptide can be used to identify LT or CT epitopes that are particularly effective at eliciting anti-LT or anti-CT antibody titers. LT or CT epitopes responsible for a strong anti-LT or anti-CT antibody response against E. coli or V. cholerae can be identified by eliciting anti-LT or anti-CT antibodies directed against LT or CT polypeptides of different lengths. Anti-LT and anti-CT antibodies elicited by a particular LT or CT polypeptide epitope can then be tested using an anti-LT or anti-CT ELISA assay to determine which polypeptides contain epitopes that are most effective at generating a strong response. LT or CT polypeptides or fusion proteins which contain these epitopes or polynucleotides encoding the epitopes can then be constructed and used to elicit a strong anti-LT or anti-CT antibody response.

A composition of the invention comprising a LT or CT polypeptide, or a combination thereof is administered in a manner compatible with the particular composition used and in an amount which is effective to elicit an immune response to LT or CT, for example an anti-LT or anti-CT antibody titer as detected by an ELISA, as described above.

An LT or CT polypeptide can be administered to an animal or human, to elicit an immune response in vivo. Oral delivery of an LT or CT polypeptide is preferred. Administration of a polypeptide can be by any means known in the art, including oral, intramuscular, intradermal, intraperitoneal, or subcutaneous injection, including injection using a biological ballistic gun (“gene gun”). Administration may also be intranasal. Preferably, an LT or CT polypeptide is accompanied by a protein carrier for oral administration. A combination of administration methods may also be used to elicit an anti-LT or anti-CT immune response. For example, one dose of the immunogenic composition may be administered by one route, such as oral, while another dose or booster may be administered by the transdermal, subcutaneous, intravenous, intramuscular, intranasal or intrarectal route.

The particular dosages of an antigenic composition of the invention will depend on many factors including, but not limited to the species, age, and general condition of the human or animal to which the composition is administered, and the mode of administration of the composition. An effective amount of the composition of the invention can be readily determined using only routine experimentation. In vitro and in vivo models described herein can be employed to identify appropriate doses. Generally, 0.1, 1.0, 1.5, 2.0, 5, 10, or 100 mg/kg of an antigen will be administered to a large mammal, such as a baboon, chimpanzee, or human. If desired, co-stimulatory molecules or adjuvants can also be provided before, after, or together with the antigenic compositions. LT and CT polypeptides of the invention can be administered either to an animal or human that is not infected with E. coli or V. cholerae or can be administered to an E. coli or V. cholerae-infected animal or human.

Immune responses, including elicitation of anti-LT and anti-CT IgG and/or IgA antibodies, of the animal or human generated by the delivery of a composition of the invention can be enhanced by varying the dosage, route of administration, or boosting regimens. Compositions of the invention may be given in a single dose schedule, or preferably in a multiple dose schedule in which a primary course of vaccination includes 1-10 separate doses, followed by other doses given at subsequent time intervals required to maintain and/or reenforce an immune response, for example, at 1-4 months for a second dose, and if needed, a subsequent dose or doses after several months. Preferably, at least one of these administrations is performed orally to elicit a mucosal immune response as well as to take advantage of cost and convenience. Oral administration comprises consuming a transgenic plant or plant part of the invention.

Administration of LT or CT polypeptides can elicit an anti-LT or anti-CT antibody titer and/or cellular immune response in the animal or human that lasts for at least 1 week, 2 weeks, 1 month, 2 months, 3 months, 4 months, 6 months, 1 year, or longer. Optionally, an anti-LT or anti-CT immune response can be maintained in an animal or human by providing one or more booster injections of the LT or CT polypeptide at 1 month, 2 months, 3 months, 4 months, 5 months, 6 months, 1 year, or more after the primary administration. Optionally, the administration of the polypeptides of the invention as plant matter or foodstuff is used as a booster after a primary injection of a composition that elicits an immune response to LT or CT.

To determine the immunogenicity of plant-derived LT or CT polypeptides of the invention when fed orally, extracts or plant tissues from plants expressing the LT or CT polypeptides of the invention can be fed to animals, such as mice or humans. For example, one group of mice can be fed an extract of a plant or a plant tissue where the plant expresses a polypeptide of the invention. Another group of mice can be given recombinant LT or CT polypeptides purified from E. coli expressing the same antigen from a recombinant plasmid. Serum and mucosal antibody responses can be examined by ELISA. See Example 21. Further, toxin neutralization assays can be performed to determine the protective nature of the antibodies elicited in, for example mice, by the plant derived samples. See WO 96/12801 and Example 21.

Use of LT and CT Polypeptides and Polynucleotides as Adjuvants

LT and CT polypeptides and polynucleotides of the invention can be used as adjuvants for other immunogenic compositions. The inclusion of LT-A, LT-B, CT-A, CT-B, LT or CT polypeptides and mutants and combinations thereof, or polynucleotides encoding these polypeptides, or combinations thereof in an immunogenic composition can facilitate the transportation into intestinal mucosa cells of gene and protein fusions of other antigens and can lead to enhanced mucosal and humoral immune responses to other antigens. The adjuvant polypeptide or polypeptides can be present as a fusion protein along with an antigen and can act as adjuvants to enhance the immune response to the fused antigens. The antigens of pathogenic microorganisms suitable for fusing with the LT and CT polypeptides of the invention include, for example, colonization antigens, virulence antigens, and epitopes of virulence antigens or colonization antigens.

For example, polypeptides encoding LT-A, LT-B, CT-A, CT-B, LT holotoxin, or CT holotoxin or mutants thereof can be fused with the Norwalk virus capsid protein (NVCP) (Jiang et al. 9 J. Virol. 66:6527 (1992)) or the hepatitis B surface antigen (HBsAg) using techniques known in the art. See Example 15 and WO96/12801. For example, an ELISA assay for NVCP can be done using rabbit anti-I-rNV as the capture antibody and guinea pig anti-I-rNV as the detector antibody to demonstrate the expression of the Norwalk virus antigen. See WO 96/12801. The immune response of animals or humans fed plants or plant extracts comprising such a fusion protein can be measured by, for example anti-LT, anti-CT and anti-NVCP ELISA assays for IgG and IgA. Immunogenic polypeptides and polynucleotides of the invention encoding LT-A, LT-B, CT-A, CT-B, LT or CT and mutants and combinations thereof can also be used as an adjuvant in animals and humans when provided orally with a separate heterologous antigen or vaccine.

Further, transgenic plants comprising both an adjuvant of the invention and a heterologous antigen can be produced by sexual crossing of a plant that expresses an antigen of the invention and a plant that expresses the heterologous antigen of interest, for example NVCP or HBsAg. The result is a progeny plant that expresses both an adjuvant of the invention and a heterologous antigen for use in the treatment or prevention of a specific disease or infection. See Example 19.

Many methods can be used to verify the mucosal adjuvant activity for plant-expressed adjuvants of the invention. For example, either the crude plant material (NT1 cells or tomato fruit) expressing an adjuvant of the invention or extracts thereof can be fed to mice along with a model antigen, ovalbumin. Immune responses (serum IgG and fecal secretary IgA) in the mice directed against ovalbumin are compared with responses in control animals that are fed ovalbumin alone. The adjuvant activity is calculated as the fold-increase in the geometric mean antibody titers against ovalbumin between the two test groups. Sufficient numbers of mice in each group (8-10) permit calculation of standard errors, which enable a determination of statistical significance of the data.

The following are provided for exemplification purposes only and are not intended to limit the scope of the invention described in broad terms above. All references cited in this disclosure are incorporated herein by reference.

### EXAMPLES

Example 1

### Construction of Synthetic LT-A (s-LTA) Encoding Sequence

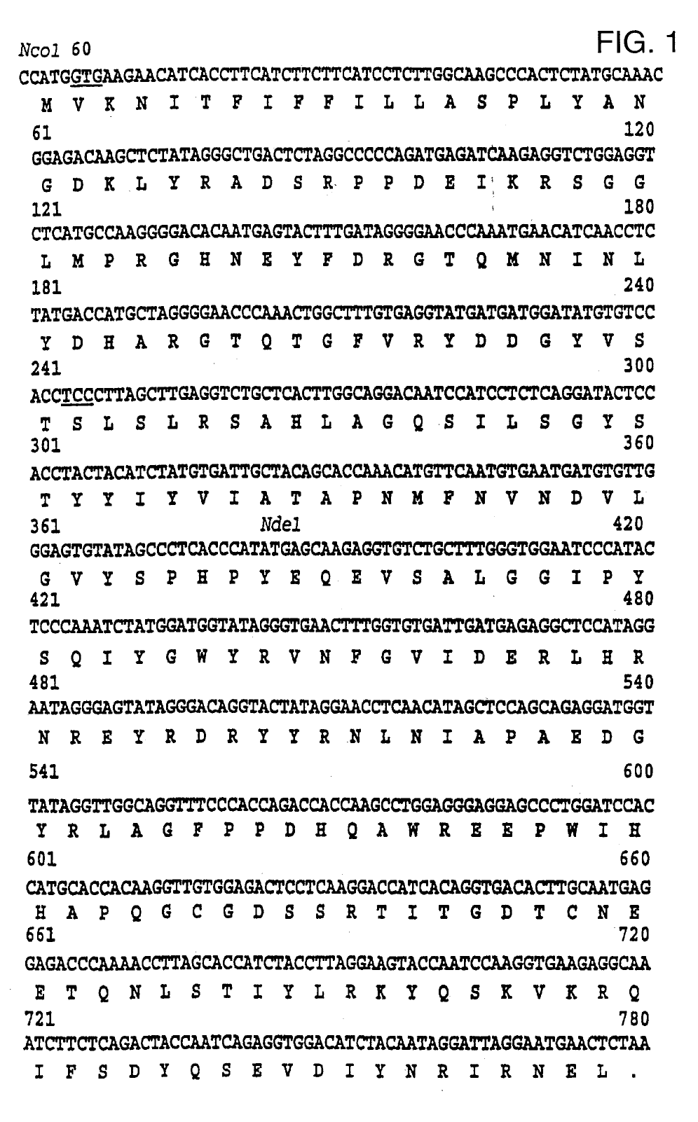

Codon use tables (Ausubel F., et al., eds. Current Protocols in Molecular Biology, vol. 3, p.A.1C.3 (1994)) were used to design the plant-preferred encoding sequence for sLT-A and to eliminate sequence motifs associated with spurious mRNA processing. Adang et al. Plant Mol Biol 21:1131-1145 (1993). A single codon insertion (GTG encoding valine) was made to accommodate the creation of a NcoI restriction site around the initiator methionine codon. FIG. 1 shows the nucleotide sequence of sLT-A (SEQ ID NO:1).

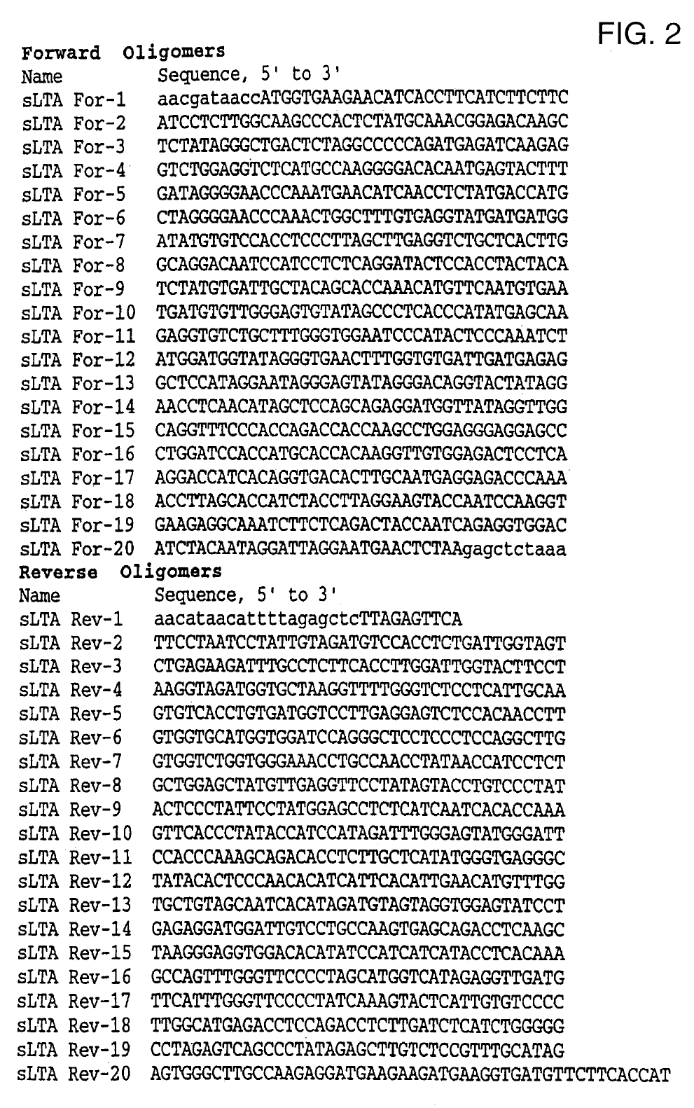

Oligonucleotide 40-mers spanning the entire sLT-A coding sequence and a set of complementary 40-mers that center on the junctions of the coding oligomers allowing 20 base pair overlaps were obtained commercially (FIG. 2) (SEQ ID NOs:2-41). NcoI and SacI sites were included at the 5′ and 3′ ends, respectively, of the coding sequence in order to facilitate cloning of the fragment into plant expression plasmids. Assembly PCR was used to assemble and amplify the coding sequence as described in Stemrner et al., Gene. 164:49-53 (1995). The final amplification product was digested with Nco 1 and Sac 1 and cloned into pGEM-5zf(+) (Promega, Madison, Wis.) to form pGEM-sLTA. Clones were selected by DNA sequencing, and one clone was obtained in which the entire sequence was verified.

### Example 2

### Site-Directed Mutation of pGEM-sLTA to make S63K Substitution

Polymerase chain reaction (PCR) was used to alter the codon underlined at position 244 (FIG. 1) (SEQ ID NO:1) from “TCC” (S) to “AAG” (K) to create the sLTA-K63 mutant. A mutagenic oligonucleotide with the sequence: 5′-GCTAAGCTTGGTGGACACATA-3′ (SEQ ID NO:49) was obtained commercially and used with the pUC/M13-Forward primer (5′-GTAAAACGACGGCCAGT-3′) (SEQ ID NO:50) to amplify a 330 bp fragment using pGEM-sLTA as template. The 330 bp product was purified by electrophoresis and used as a “megaprimer” along with the pUC/M 13-Reverse primer (5′-AACAGCTATGACCATG-3′) (SEQ ID NO:51) to amplify the full sLTA-K63 coding sequence using pGEM-sLTA as template. The sLTA-K63 PCR fragment was cloned into T-tailed pBluescript-KS (Stratagene, La Jolla, Calif.) and the product sequence was confirmed between the NcoI site at the 5′ end and the NdeI site at position 380 (FIG. 1) (SEQ ID NO:1). The NcoI/NdeI fragment of sLTA-K63 was then substituted for the same fragment in pGEM-sLTA to form pGEM-sLTA-K63.

### Example 3

### Generation of G192 Mutant in sLT-A

The LT-A cleavage site mutant Arg 192 to Gly 192 (R192G) is reported to be a low toxicity mucosal adjuvant (Dickinson & Clements, 1995, 1998). Because of the potential for plant cell expression of holotoxin LT-G192, the G192 mutation was studied in the plant-optimized sLTA gene.

The G192 mutation was created by the “megaprimer” PCR method as described above for sLTA-K63. A primer was designed that incorporated a single base change (A to G) at position 631 of the sLT-A gene in pGEM-sLTA, thus changing the AGG (Arg) codon to GGG (Gly). The primer with the altered nucleotide underlined had the sequence: 5′-CTCAGGGACCATCACA-3′ (SEQ ID NO:52). This primer was used along with the pUC/M13-Reverse primer to amplify a 233 bp fragment using pGEM-sLTA as a template. The gel-purified 233 bp product was used as a “megaprimer” along with the sLTA-F1 primer (SEQ ID NO:2) to amplify the full sLTA-G192 coding sequence using pGEM-sLTA as template. The sLTA-G192 PCR fragment was cloned into T-tailed pBluescript-KS (Stratagene, La Jolla, Calif.) and sequenced to verify as correct, and named pGEM-sLTA-G192.

### Example 4

### Generation of R72 Mutant in Synthetic LT-A Gene

Mutations in the enzyme active sites of A subunits of cholera toxin and E. coli heat labile toxin LT limit the toxicity of these proteins while maintaining adjuvant activity. (Fontana et al, 1995); De Magistris (1996) Mucosal Immunization: Genetic Approaches & Adjuvants. IBC Biomedical Library, Southborough, Mass., pp. 1.8.1-1.8.12 (Based on a presentation at the IBC Conference, Oct. 16-18, 1995, Rockville, Md.). The LTA-R72 mutant was shown to have lowered but measurable enzymatic activity; when assembled with the LT-B pentamer (LT-R72), it is reportedly a more potent mucosal adjuvant than LT-K63 (Rappuoli, 1998). In order to obtain further evidence that active site mutants of LT-A can be expressed efficiently in plant cells, the Ala72 to Arg72 (A72R) mutant of the synthetic plant-optimized LT-A (sLT-A) gene was prepared.

In order to introduce the R72 mutation into the sLT-A gene, the nucleotides at positions 271 and 272 (GC) were changed to AG, thus changing the codon GCA (Ala) to AGA (Arg). To effect this change, the QUIKCHANGE™ site-directed mutagenesis kit (Stratagene, La Jolla, Calif.) was used with the mutagenic primer set:

5′-AGGGTCTGCTCACTTGAGAGGACAATCCATCCTC-3′ (SEQ ID NO:53)

3′-TCCCAGACGAGTGAACTCTCCTGTTAGGTAGGAG-5′ (SEQ ID NO:54)

The underlined nucleotides show the sites of mutation. Using the methods specified in the kit, PCR was used to amplify the plasmid pGEM-sLTA using Pfu DNA polymerase. The reaction parameters were: 1 cycle at 95° C. for 30 sec, followed by 16 cycles of (95° C. for 30 sec, 55° C. for 1 min, 68° C. for 7.5 min). The reaction products were digested with DpnI restriction enzyme according to kit instructions, and then used to transform E. coli XL-1 Blue cells (Stratagene). Plasmids were prepared from transformant colonies and screened by digestion with MwoI restriction endonuclease. Mutants were distinguished by the loss of a MwoI site in the sLT-A sequence. Positive clones were sequenced fully from both ends of the sLT-A gene in order to verify the mutations and an otherwise correct sequence, and named pGEM-sLTA-R72.

### Example 5

### K63/G192 and R72/G192 Double Mutants in sLT-A.

Double mutants of the plant-optimized sLTA gene containing either the K63 and G192 substitutions (K63/G192) or the R72 and G192 substitutions (R72/G192) can be generated. The 3 80 bp NcoI/NdeI fragment of psLTA-K63 is substituted for the same fragment in psLT-A-G192 to give psLTA-K/G. Transformants are screened by digestion of plasmids with HindIII in order to identify the HindIII site contained in the K63 mutation that is not present in psLTA-G192. Recombinants are sequenced to verify the correct DNA sequence.

Similarly, the 380 bp NcoI/NdeI fragment of psLTA-R72 is substituted for the same fragment in psLTA-G192 to give psLTA-R/G. Transformants are screened by digestion of plasmids with MwoI in order to identify clones lacking the MwoI site contained in psLTA-G192, which is not present in psLTA-R/G. Recombinants are sequenced to verify the correct DNA sequence.

### Example 6

### Construction of Expression Plasmids

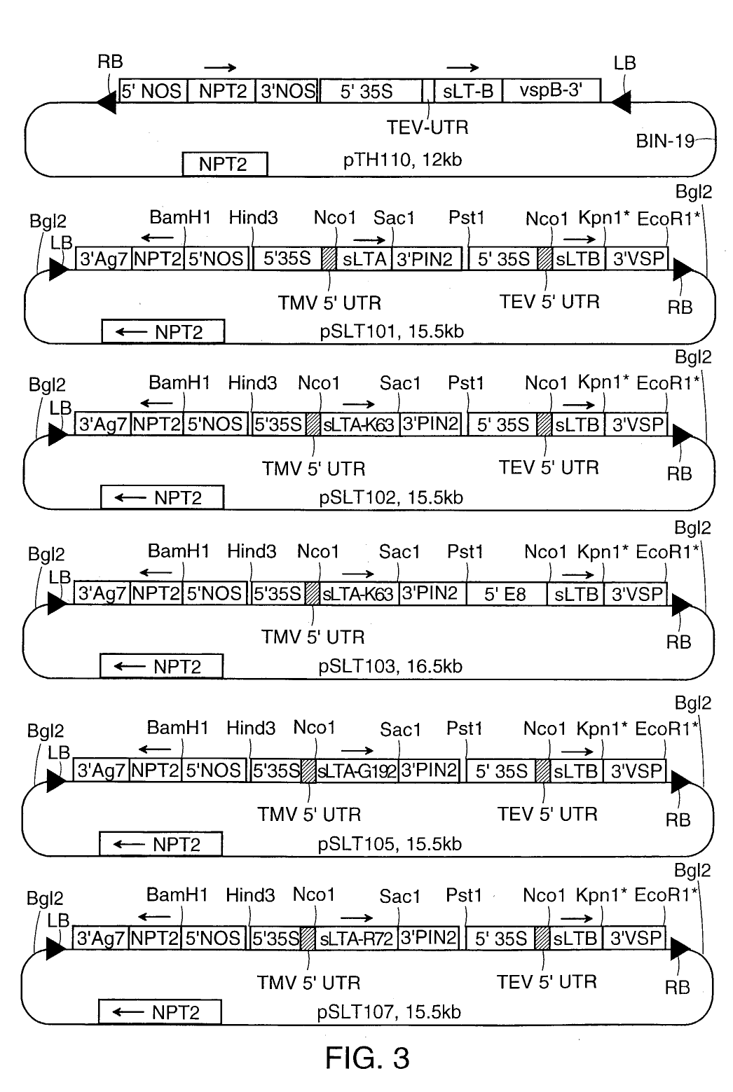

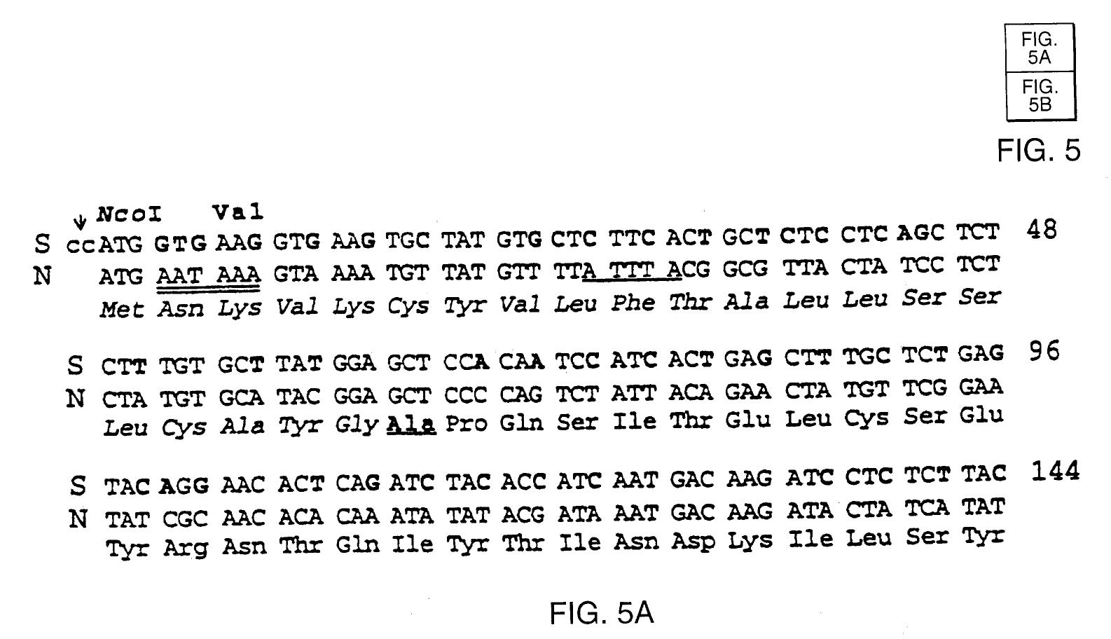

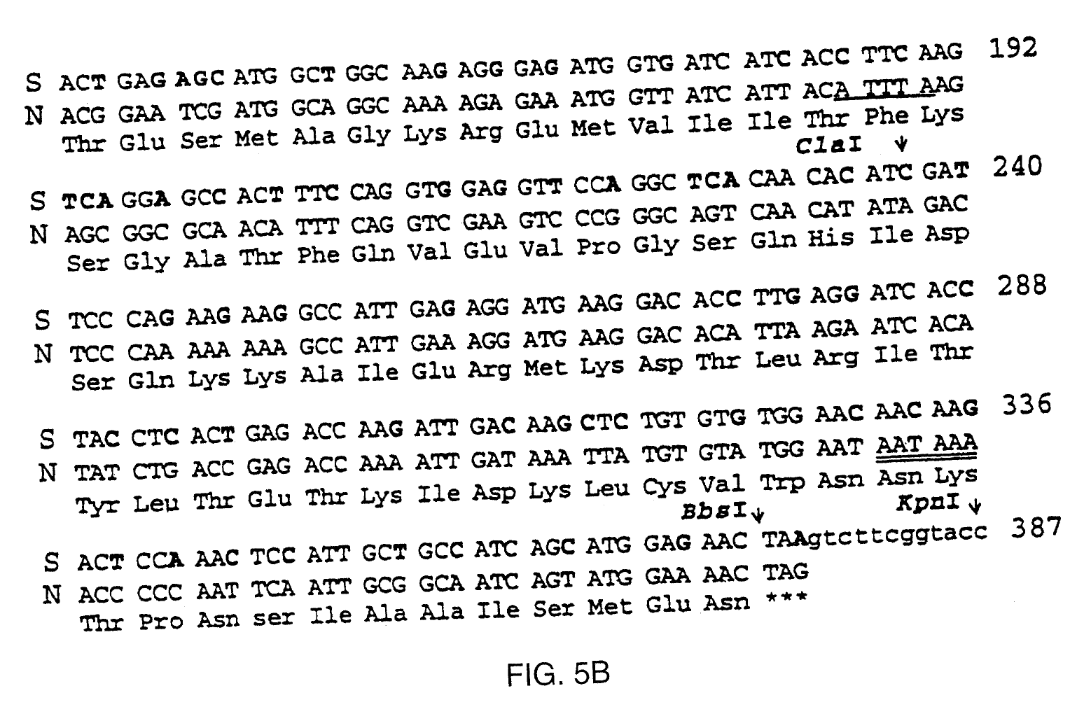

pTH110, pTH210: PTH110 (FIG. 3, FIG. 5, Mason et al, 1998) is an expression plasmid for sLT-B, which contains the sLT-B expression cassette from pTH210 cloned into pB1101 (Clontech, Palo Alto, Calif.) between HindIII and EcoRI. pTH210 is identical to pLTB210 (Haq et al., 1995) except that the coding region is plant-optimized (Mason et al., 1998).

pSLT101: The NcoI/SacI fragments containing the full wild-type or mutant LT-A coding sequences of pGEM-sLTA and pGEM-sLTA-K63 were purified and ligated into pBT1211 to form pSLTA211 and pSLTA(K63)211. Plasmid pBT1211 contains the double enhancer 35S promoter fused to the tobacco mosaic virus “ω” UTR (Gallie et al., Nucleic Acids Res. 20:4631-4638 (1992). followed by a polylinker and the vspb terminator (Haq et al. 1995). An EcoRV/XhoI fragment from plasmid pBSG660 (Biosource Technologies, Inc., Vacaville, Calif.) containing the 3′ end of the 35S promoter and the “ω” UTR was subcloned into the EcoRV/XhoI sites of pIBT210 (Haq et al. 1995) to create the intermediate pBT1211.1. In order to delete the TEV leader, pBT1211.1 was digested with XhoI and NcoI, followed by mung bean nuclease, to blunt the ends, and blunt ligation. The resulting pBT1211 is similar to pIBT210 (Haq et al. 1995), except that the tobacco mosaic virus “ω” UTR is substituted for the TEV 5′-UTR.

The binary T-DNA plasmid pSLT10 (FIG. 3) was constructed by ligation of four DNA fragments: a 10 kb vector pGPTV-KAN (Becker, et al Plant Mol. Biol. 20:1195-1197 (1992)) digested with HindIII and EcoR1; a 1.8 kb fragment containing the sLT-A gene under control of a 35S promoter from digestion of pSLTA211 with HindIII and SacI; a 1 kb fragment containing the PIN-2 termination sequence from digestion of pRT38 (Thornburg et al., Proc. Natl. Acad. Sci. USA. 84:744-748 (1987)) with SacI and PstI; and a 2 kb fragment containing the sLT-B gene flanked by the 35S promoter and a VSP termination sequence from digestion of pTH210 with PstI and EcoRI. pSLT102: This plasmid was constructed in the same way as pSLT101 except that the 1.8 kb fragment from digestion of pSLTA(K63)211 with HindIII and SacI was used instead of that from pSLTA211.

pSLT103: A binary vector for expression of LT-K63 in tomato fruit was constructed. To make pSLT103, an intermediate clone, pE8TH was made by ligation of a 2 kb EcoRI/NcoI fragment containing the tomato E8 promoter (Giovannoni et al. Plant Cell. 1:53-63 (1989)) with a 0.4 kb NcoI/KpnI fragment containing the sLT-B coding sequence from pTH210 and pBluescript KS digested with EcoRI and KpnI. pSLT103 was assembled from four DNA fragments: 10.6 kb vector pSLT101 digested with HindIII and KpnI, a 1.8 kb fragment from digestion of pSLTA(K63)211 with HindIII and SacI, 1 kb fragment from digestion of pRT38 with SacI and PstI, and 2.4 kb fragment from digestion of pE8TH with PstI and KpnI. The construct therefore contains the sLT-A-K63 gene driven by the constitutive 35S promoter, while the sLT-B gene is driven by the fruit-specific E8 promoter (FIG. 3).

pSLT105: In order to create a coexpression plasmid vector with the sLT-A-G192—gene, the NcoI-SacI fragment containing the verified sLTA-G192 sequence is subcloned into pBT1211 to make psLTA-G192-211, in which the sLTA-G192 gene is flanked by the CaMV 35S promoter and TMV ω leader on the 5′ side. Then, the 1.8 kb promoter-leader—sLTA-G192 fragment is obtained by digestion of psLTA-G192-211 with HindIII and SacI, and ligated as described for pSLT101 to give pSLT105 (FIG. 3). Thus, pSLT105 is identical to pSLT101 except that the sLTA-G192 coding sequence is substituted for the sLTA coding sequence.

pSLT107: In order to create a coexpression plasmid vector using the sLTA-R72 gene, the coding sequence was obtained by digestion of pGEM-sLTA-R72 with NcoI and SacI, and the 780 bp fragment purified by agarose gel electrophoresis. This fragment was ligated with pBTI1211/NcoI-SacI to give psLTA-R72-211, in which the sLTA-R72 gene is flanked by the CaMV 35S promoter and TMV ω leader on the 5′ side. Then, the 1.8 kb promoter-leader-sLTA-R72 fragment was obtained by digestion of psLTA-R72-211 with HindIII and SadI, and ligated as for pSLT101 to give pSLT107. Thus, pSLT107 is identical to pSLT11 except that the sLTA-R72 coding sequence is substituted for the sLTA coding sequence.

### Example 7

### Transformation of Tobacco NT1 Cells

NT1 cells are a non-photoautotrophic callus cell line of Nicotiana tabacum that can be maintained either on solid media or in liquid suspension. An G, Plant Physiol. 79:568-570 (1985). The cells are grown at 26 to 28° C. on a rotary shaker (150 rpm) in a 250 mL Erlenmeyer flask containing 40 mL of NT medium (MS salts, 30 g/L sucrose, 3 μM thiamine, 0.56 mM myoinositol, 1.3 mM KH₂PO₄, 1 μM 2,4-D, 2.5 mM Mes, pH 5.7) and subcultured using 5% inoculum every 7 days. The cells can also be grown at lower temperatures, but the growth kinetics are altered and subculture at longer intervals are used, e.g., 8 to 10 days for culture at 23° C.

T-DNA plasmids are used to transform A. tumefaciens LBA4404 or EHA 105 by electroporation, using the same method commonly used for E. coli. The Agrobacterium strain containing the expression construct is grown in liquid medium by inoculation of a single colony from a freshly streaked plate (LB-agar+50 mg/L kanamycin) into 5 mL of YM (Life Technologies 10090-017; per L: 0.4 g yeast extract, 10 g mannitol, 0.1 g NaCl, 0.2 g MgSO₄·7H₂O, 0.5 g K₂HPO₄) with 50 mg/L kanamycin, and grown at 30° C. with vigorous shaking for 12 to 16 hours. The O.D. of the culture at 600 nm is determined and the culture is stopped at O.D. of 0.5. If the culture has overgrown, it is diluted in fresh YM+50 mg/L kanamycin and grown at 30° C. until the O.D. at 600 nm is 0.5.

For transformation of NT1 cells (Newman et al., Plant Cell. 5:701-714 (1993)), 20 μM acetosyringone is added to the NT1 cell culture 3 days (culture at 26° C. ) or 4 days (culture at 23° C.) after subculture, and the cells are abraded by repeated (20 times) pipetting with a 10 mL plastic disposable pipet. Agrobacterium cells (10 to 20 μL of a culture at O.D. 600 nm=0.5) are mixed with 4 mL of the abraded NT1 cells and the mixture incubated in a 50 mm plastic petri dish wrapped in foil to exclude light, with shaking at 50 rpm at 23° C. for 3 days. The cells are then transferred to a 50 mL plastic conical centrifuge tube with 45 mL NT1 medium containing 500 mg/L carbenicillin (NTC), mixed and centrifuged at 1,000 rpm in a clinical centrifuge equipped with a swinging bucket rotor (Timentin, made by SmithKline/Beecham, can be substituted for carbenicillin). The pelleted cells are washed twice more with 50 mL NTC, and resuspended in 4 mL NTC. Two mL of the resuspended cells are plated on each of 2 petri plates (95 mm) containing NT agar medium with 100 to 200 mg/L kanamycin and 500 mg/L carbenicillin (NTCK). More effective selection may be obtained using 200 mg/L kanamycin. After 3 to 4 weeks, the transformed kanamycin-resistant calli are selected and subcultured on fresh NTCK agar. When calli are of sufficient size (e.g. 5 to 10 mm diameter), they can be screened for transgene presence by PCR, or for expression by ELISA, Western blot, or Northern blot.

NT1 cell lines were transformed with expression plasmids of the invention using Agrobacterium tumefaciens strain LBA4404 electroporated with the synthetic binary plasmid. Four day old cultures of NT-1 cells were treated with acetosyringone and pipetted up and down in order to induce lesions that would improve transformation efficiency. The NT1 cells were then cocultivated with 25-100 μL aliquots of an overnight sLT101 or sLT102 Agrobacterium culture in 5 cm petri dishes. Cocultivation dishes were incubated for three days after which cells were washed extensively with NTKC media and plated on NT1 plates supplemented with kanamycin and carbenicillin. In general, transformants are expected to appear three to four weeks after this date.

### Example 8

### Extraction of Tobacco Cells

Samples of tobacco cells, either from solid medium or liquid suspension cultures, were placed in 1.5 mL microcentrifuge tubes and frozen on dry ice immediately. Liquid suspension cultured cells were collected by centrifugation at 3,000×g for 5 min, and the supernatant medium saved for ELISA to examine secretion of recombinant protein into the medium. The cell volume was estimated and 3 volumes of extraction buffer (25 mM sodium phosphate, pH 6.6; 50 mM sodium ascorbate; 100 nM NaCl; 1 mM EDTA; 1% Triton X-100; 10 μg/mL leupeptin) were added to the frozen cells. A plastic pestle shaped to fit the microcentrifuge tube and attached to a drill was used to homogenize the cells in buffer. The samples were kept on ice for 10 min, and then centrifuged at 16,000×g for 5 min. The supernatant was kept for ELISA.

### Example 9

### Ganglioside-Dependant ELISA for LT-B and Mutant LT-A

As shown in Table 2, an ELISA was performed for ganglioside binding-dependent detection of LT-B as described by Cardenas & Clements (Infect. Immun. 61:4629-4636 (1993)), except that the detector antibody was goat anti-LT holotoxin (gift of Dr. John Clements, Tulane Medical Center, New Orleans, La.) instead of anti-LT-B. To detect assembled LT holotoxin, the same ELISA was modified by use of a detector antibody specific for CT-A (gift of Dr. John Clements, Tulane Medical Center, New Orleans, La.), which cross-reacts with LT-A but not LT-B. Because LT-A does not bind to ganglioside unless assembled into holotoxin with LT-B,—the ganglioside-dependent ELISA for LT-A proves holotoxin assembly. Samples of cell extracts or medium from suspension cells were diluted in 1% nonfat dry milk in phosphate-buffered saline (PBS), pH 7.2 for ELISA.

TABLE 2
ELISA detection of LT-B and assembled LTA-K63 in tobacco cells
ng/μg total soluble protein

| Cell line | α-LT-B | α-CT-A |
|---|---|---|
| NT1 | 0.0 | 0.0 |
| NT1/TH110-1 | 1.1 | 0.0 |
| NT1/TH110-2 | 1.5 | 0.0 |
| NT1/SLT102-1 | 1.4 | 2.7 |
| NT1/SLT102-2 | 2.2 | 5.8 |

The standard curve was constructed using dilutions of LT (gift of Dr. John Clements, Tulane Medical Center, New Orleans, La.) between 3.1 and 100 ng/mL. Total protein in extracts was determined by the Bradford assay (BioRad) with bovine serum albumin (BSA) as a standard, and used as a basis for the presentation of ELISA results.

Further, the culture medium from suspension cultured NTI/SLT102 cells contained a substantial proportion of the total LT in the culture (data not shown). This observation strongly suggests that at least some of the recombinant protein is secreted into the medium.

### Example 10

### Expression of LT Holotoxin in NT1 Cell Lines Transformed with pSLT105 and pSLT107

This example shows that the G192 and R72 mutant forms of LT-A can be expressed in transgenic tobacco NT1 cells, and that each assembles with LT-B expressed in the same cells to produce a ganglioside-binding holotoxin complex.

NT1 tobacco cells were stably transformed with pSLT105 (G192) and pSLT107 (R72), which contain expression cassettes for mutant LT-A forms (G192 or R72) and LT-B. Coexpression of LT-A in the same cells with LT-B is expected to allow assembly of A₁:B₅ holotoxin complexes. Assembled holotoxin can bind ganglioside G_{M1}, via the LT-B pentamer, but unassembled LT-A is not expected to bind ganglioside. Thus, the presence of assembled holotoxin can be assayed using the ganglioside-dependent ELISA and an antibody probe that is specific for LT-A (unreactive with LT-B). As described in Example 9, ganglioside-dependent ELISA was performed using anti-LT (holotoxin) and anti-cholera toxin A subunit (CT-A), which cross-reacts with LT-A but not LT-B (Table 3). LT holotoxin expressed in E. coli cells was used as the standard for both probes.

NT1 cell clumps were gown on solid agar medium containing 100 μg/ml kanamycin, and were extracted in modified PBS as described before. Dilutions of the extracts were assayed by ganglioside-dependent ELISA using anti-LT and anti-CT-A probes, and for total soluble protein by the Bradford assay (BioRad kit) using BSA as a standard. Determinations of LT (anti-LT probe) and LT-A in assembled A₁:B₅ holotoxin complexes (anti-CT-A probe) were normalized to total soluble protein to obtain ng recombinant protein per μg total soluble protein. The results shown Table 3 indicate expression of LT in independent transgenic lines varies considerably, as expected for Agrobacterium-mediated DNA delivery. However, as observed previously with pSLT102 transformants, the apparent concentration of LT-A in assembled A₁:B₅ holotoxin complexes was always higher than that of LT measured with anti-LT. The reason for this observation is unclear, but may indicate that the plant-produced LT-A is more reactive with anti-CT-A than bacterial LT-A.

TABLE 3
ng antigen per microgram total protein

| Cell line | LT (anti-LT) | LT-A in holotoxin (anti-CT-A) |
|---|---|---|
| SLT105-24 | 1.5 | 2.6 |
| 58 | 0.2 | 0.6 |
| 88 | 0.9 | 1.9 |
| 141 | 0.7 | 1.1 |
| 144 | 0.8 | 2.6 |
| SLT107-4 | 2.0 | 9.6 |
| 15 | 0.7 | 5.8 |
| 18 | 0.8 | 4.6 |
| 30 | 1.6 | 14.7 |
| 32 | 1.2 | 10.6 |
| 33 | 0.4 | 9.8 |
| 73 | 0.7 | 5.8 |

### Example 11

### Construction of LT-B Gene Containing a Plant Signal Peptide and Expression in Tobacco NT1 Cells

This example describes the construction of a modified gene encoding E. coli heat-labile enterotoxin B subunit LT-B), wherein the bacterial signal peptide is replaced by a plant-derived signal peptide from soybean vspA. Mason et al., Plant Mol. Biol. 11:845-856 (1988). Although the bacterial signal peptide appears to function normally in plants, yielding assembled ganglioside-binding LT-B pentamers, a plant signal peptide may allow improvement of protein delivery to the endoplasmic reticulum (ER) and thus enhanced accumulation of assembled pentamers. This example further describes use of the modified gene to create transgenic tobacco NT1 cells and demonstrates that correctly assembled ganglioside-binding LT-B pentamers are formed in these cells.

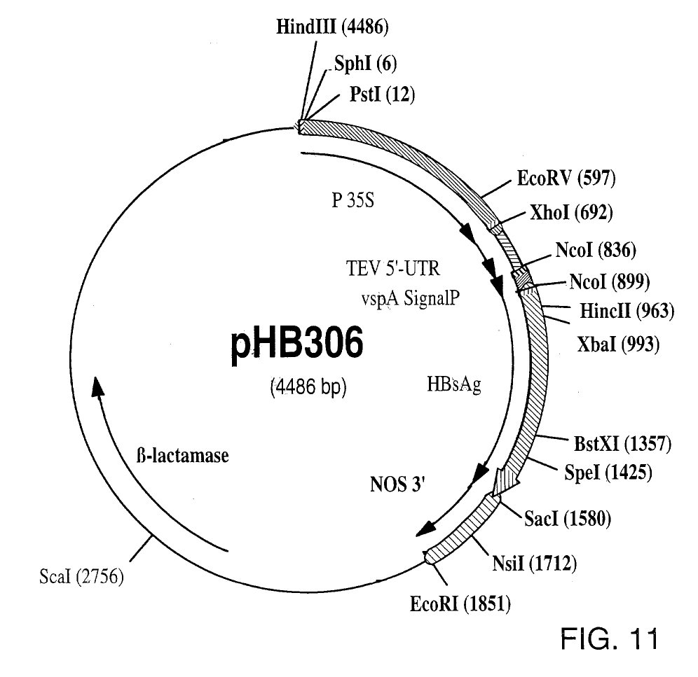

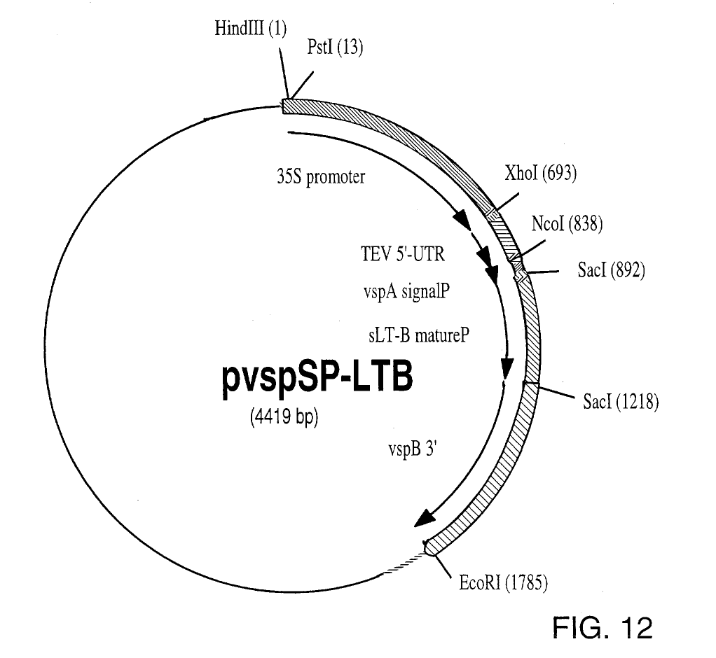

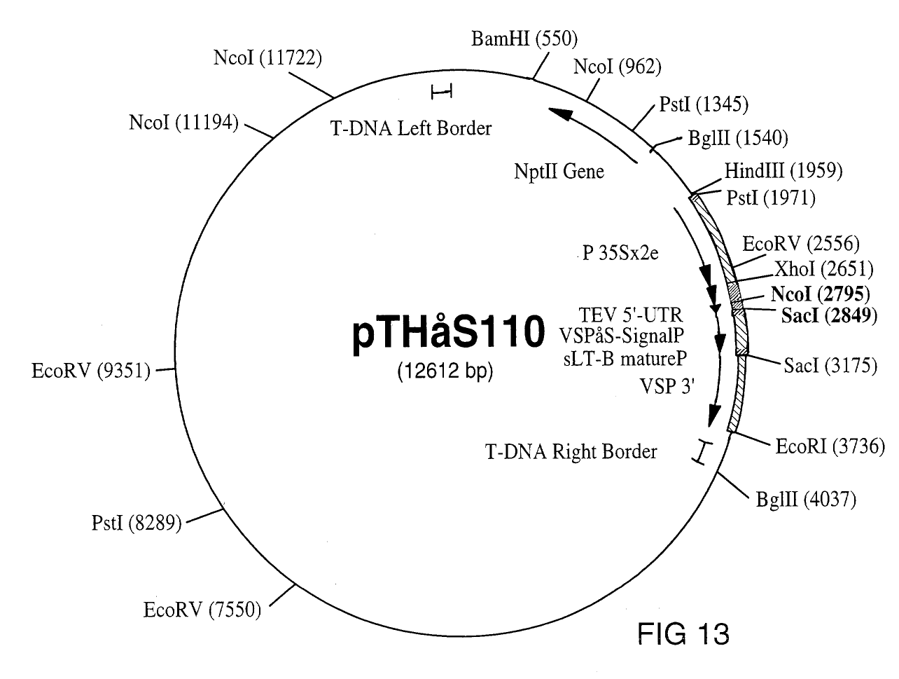

Primer S-VSP-Sac with sequence 5′-CTGGAGCTCCCCATGCTACCAAAAT-3′ (SEQ ID NO:55) and primer 35S with sequence 5′-AATCCCACTATCCTTCG-3′ (SEQ ID NO:56) were used with the template pHB306 (FIG. 11) to amplify a fragment of approximately 260 bp containing the soybean vspA signal peptide (DeWald, D. Ph.D. Dissertation, Dept. of Biochemistry & Biophysics, Texas A&M University, College Station, Tex., 1992). pHB306 contains the soybean vspA signal peptide fused to the hepatitis B surface antigen in the expression vector pIBT211.1. The resulting PCR product was digested with XhoI and SacI to obtain the 199 bp fragment containing the TEV 5′-UTR and the vspA signal peptide. This fragment was inserted into the XhoI and SacI sites of pTH210 (Haq et al., Science 1995; 268:714-716) to yield pvspSP-LTB (FIG. 12). Finally, the HindIII/EcoRI fragment of pvspSP-LTB containing the entire expression cassette was ligated with pGPTV-KAN to produce pTHαS110 (FIG. 13).

Tobacco NT1 cells transformed with pTHαS110 as described in Example 7, and selected stably transgenic cell lines on media containing kanamycin. Individual lines were assayed for LT-B by ganglioside-dependent ELISA as described in Example 9. This assay detects only assembled LT-B pentamers that can bind the natural ligand ganglioside G_{M1}. The results in Table 4 show the results for the best 8 lines of 30 lines screened. Levels of LT-B in these cell lines ranged up to 3.1 ng per g total soluble protein (TSP). These levels are comparable to and in some cases higher than levels obtained with pTH110, containing the bacterial signal peptide (Example 9).

In conclusion, the vspA signal peptide was fused to the plant-optimized LT-B sequence encoding the mature peptide. The recombinant gene directed expression of ganglioside-binding LT-B pentamers in tobacco NT1 cells. Thus the vspA signal peptide is effective in targeting LT-B to the ER and causing the correct folding and assembly of functional LT-B pentamers. A more precise comparison of the effectiveness of the bacterial or vspA signal peptides with LT-B could be performed using pulse-chase cell labeling with radioisotopically labeled amino acids in order to determine the rates of synthesis, assembly, and degradation of the LT-B polypeptides. A similar construction with the plant-optimized LT-A gene using a plant signal peptide can be performed to test the idea that expression of LT-A could be improved with a plant signal peptide.

TABLE 4
LT-B in NT1 cell lines transgenic for pTHαS110

| Line | LT-B, ng per μg TSP |
|---|---|
| 5 | 2.5 |
| 9 | 0.6 |
| 14 | 1.2 |
| 16 | 2.7 |
| 24 | 3.1 |
| 25 | 1.2 |
| 26 | 0.8 |
| 27 | 1.6 |

### Example 12

### Expression of Holotoxin in Potato Transformed with pSLT102

This example shows that the K63 mutant form of LT-A can be expressed in leaves of transgenic potato plants, and that it assembles with LT-B expressed in the same cells to produce a ganglioside-binding holotoxin complex. This observation illustrates that production of assembled A₁:B₅ holotoxin complexes can be achieved in differentiated tissues of whole plants.

Potato (Solanum tubersum L. cv. “Desiree”) plants were stably transformed with pSLT102 which contains expression cassettes for mutant LT-A (K63) and LT-B. The potato plants were transformed by Agrobacterium-mediated DNA transfer essentially as described (Mason et al., 1998). Regenerated transgenic shoots were rooted on medium containing 50 μg/ml kanamycin. Leaves of plantlets propagated in agar medium were excised and extracted in modified PBS as described (Mason et al., 1998). Dilutions of the extracts were assayed by ganglioside-dependent ELISA using anti-LT and anti-CT-A probes, and for total soluble protein by the Bradford assay (BioRad kit) using BSA as a standard. Determinations of LT (anti-LT probe) and LT-A in assembled A₁:B₅ holotoxin complexes (anti-CT-A probe) were normalized to total soluble protein to obtain ng recombinant protein per μg total soluble protein.

The results shown in Table 5 indicate expression of LT in independent transgenic lines varies considerably, as expected for Agrobacterium-mediated DNA delivery. In all cases where LT is detected with the anti-LT probe, LT-A assembled as holotoxin is also observed with the anti-CT-A probe. Thus, assembled A₁:B₅ holotoxin complexes in active form can be produced in differentiated tissues of whole plants.

TABLE 5
ng antigen per microgram total protein

| Potato line | LT (anti-LT) | LT-A in holotoxin (anti-CT-A) |
|---|---|---|
| SLT102-3 | 1.0 | 0.7 |
| 14 | 0.4 | 0.4 |
| 17 | 0.2 | 0.4 |
| 25 | 0.5 | 0.4 |
| 44 | 0.1 | 0.2 |
| 78 | 0.7 | 0.3 |

### Example 13

### Holotoxin mLT-K63 Expression and Assembly in Tubers of Potato Transformed with pSLT102

This example describes characterization of the mLT-K63 produced in tubers of potato plants transformed with pSLT102 as described in Example 12. A₁B₅ complexes are assembled in the tubers, as determined by ganglioside-binding ELISA with antibody specific for LT-A.

Transgenic potato plant “Desiree”/SLT102-14 was transplanted to soil, and grown to maturity in the greenhouse as described by Mason et al., Vaccine 1998; 16:1336-1343. The tubers were harvested, washed, and air-dried. Samples of skinless tuber tissue were obtained as described by Mason et al., Vaccine 1998; 16:1336-1343, and extracted with 4 ml per gram tuber of buffer containing 25 mM sodium phosphate, pH 6,6, 100 mM NaCl, 1 mM EDTA, 1 mM PMSF, and 0.1% Triton-X-100, in a Ten-Broek ground-glass homogenizer (0.15 mm clearance). Extracts were centrifuged at 16,000×g for 3 minutes at 4° C., and dilutions of the extracts were assayed by ganglioside-dependent ELISA using anti-LT and anti-CT-A probes as described in Example 9, and for total soluble protein by the Bradford assay (BioRad kit) using BSA as a standard. Determinations of LT-B (anti-LT probe) and holo-LT (hLT) (anti-CT-A probe) were normalized to total soluble protein to obtain ng recombinant protein per μg total soluble protein (TSP), or normalized to sample fresh weight to obtain μg recombinant protein per g fresh tuber.

The data below in Table 6 show that LT-B accumulated in tubers of transgenic line SLT 102-14 to levels up to 0.19% of TSP or 8.7 μg per gram fresh tuber tissue (without skin). Further, assembled hLT-K63 accumulated up to 0.6% of TSP and 2.6-8.7 μg per gram fresh tuber. There was some variability between the two different tubers tested; however, more testing is needed to statistically determine the extent of variation. Although the smaller tuber yielded higher levels of LT-B and hLT-K63 in this case, further testing will determine whether tuber size may be a basis for antigen variability.

TABLE 6
LT-B and hLT in tubers of SLT102-14 potato plants

| | | ng per μg TSP | | μg per g tuber | | |
|---|---|---|---|---|---|---|
| Line | tuber mass | LT-B | hLT | LT-B | hLT | % Assembly |
| SLT102-14 | 42.9 | 1.3 | 0.3 | 6.0 | 1.4 | 23.3% |
| SLT102-14 | 16.5 | 1.9 | 0.6 | 8.7 | 2.6 | 30.2% |
| Desiree | 51.3 | 0.0 | 0.0 | 0.0 | 0.0 | NA |

In conclusion, SLT102 potato tubers produce LT-B subunits that efficiently assembled into ganglioside-binding pentamer form, accumulating up to approximately 8.7 μg per gram tuber. Assembled hLT-K63 accumulated in these tubers at levels up to 2.6 μg per gram fruit. Thus, potato is a potentially useful system for production of mutant LT forms for use as orally delivered mucosal adjuvant for co-delivered heterologous antigens, as well as a vaccine antigen for prevention of ETEC diarrhea. Further studies with alternative promoters, e.g. tuber specific or chemically inducible promoters, may allow enhanced levels of mutant LT protein in potato tubers.

### Example 14

### Expression of Holotoxin in Potato Transformed with pSLT107

This example shows that the R72 mutant form of LT-A can be expressed in leaves of transgenic potato plants, and that it assembles with LT-B expressed in the same cells to produce a ganglioside-binding holotoxin complex. This observation illustrates that production of assembled A₁:B5 holotoxin complexes using LT-A-R72 can be achieved in differentiated tissues of whole plants.

Potato (Solanum tubersum L. cv. “Desiree”) plants were stably transformed with pSLT107, which contains expression cassettes for mutant LT-A-R72 and LT-B. The potato plants were transformed by Agrobacterium-mediated DNA transfer as described by Mason et al., Vaccine 1998; 16:1336-1343. Regenerated transgenic shoots were rooted on medium containing 50 μg/ml kanamycin, and rooted shoots were screened for expression of LT-B. Leaves of plantlets propagated in agar medium were excised and extracted in modified PBS as described by Mason et al., 1998. Dilutions of the extracts were assayed for LT-B by ganglioside-dependent ELISA using anti-LT serum, and for total soluble protein by the Bradford assay (BioRad kit) using BSA as a standard. Based on expression of LT-B in leaves, transgenic line SLT107-28 was selected for further study, cloned, and grown to maturity in the greenhouse.

The tubers of line SLT107-28 were harvested, washed, and air-dried. Samples of skinless tuber tissue were obtained as described by Mason et al., 1998, and extracted with 4 ml per gram tuber of buffer containing 25 mM sodium phosphate, pH 6.6, 100 mM NaCl, 1 mM EDTA, 1 mM PMSF, and 0.1% Triton-X-100, in a Ten-Broek ground-glass homogenizer (0.15 mm clearance). Extracts were centrifuged at 16,000×g for 3 minutes at 4° C., and dilutions of the extracts were assayed for assembled holotbxin (hLT-R72) by ganglioside-dependent ELISA using anti-CT-A serum as described in Example 9, and for total soluble protein by the Bradford assay (BioRad kit) using BSA as a standard. Determinations of hLT-R72 were normalized to sample fresh weight to obtain μg recombinant protein per g fresh tuber. The data below in Table 7 show that hLT-R72 accumulated in tubers of transgenic line SLT107-28 up to 1.1 μg per gram fresh tuber tissue (without skin), while tubers of nontransgenic “Desiree” potato plants showed no expression.

TABLE 7
hLT-R72 expression in tubers of SLT107-28 potato plants

| Line | tuber mass, g | hLT, ng per μg TSP |
|---|---|---|
| SLT107-28 | 95.6 | 1.1 |
| Desiree | 51.3 | 0.0 |

In conclusion, SLT107 potato tubers produce LT-A-R72 and LT-B subunits that efficiently assembled into ganglioside-binding AIB, form, accumulating hLT-R72 up to 1.1 μg per gram tuber. Thus, potato is a potentially useful system for production of mutant LT forms for use as orally delivered mucosal adjuvant for co-delivered heterologous antigens, as well as a vaccine antigen for treatment or prevention of ETEC diarrhea. Further studies with alternative promoters, e.g. tuber specific or chemically inducible promoters, may allow enhanced levels of mutant LT protein in potato tubers.

### Example 15

### Co-expression of mLT and Heterologous Antigens in Transgenic Potato Tuber

This example describes co-expression of mLT's with heterologous vaccine antigens in potato tubers by creation of transgenic plants that contain expression cassettes for the plant-optimized LT-A gene (and mutants thereof), the plant-optimized LT-B gene, and at least one other antigen. Such transgenic tubers constitute an improved oral delivery system for the heterologous vaccine antigen due to the benefit of co-delivery with the mucosal adjuvant mLT.

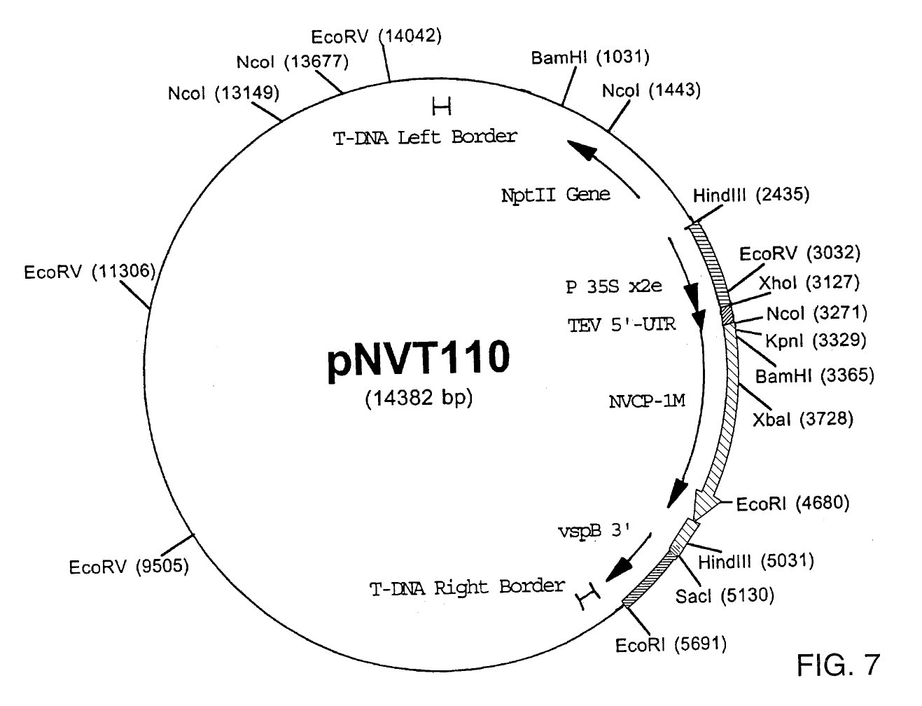

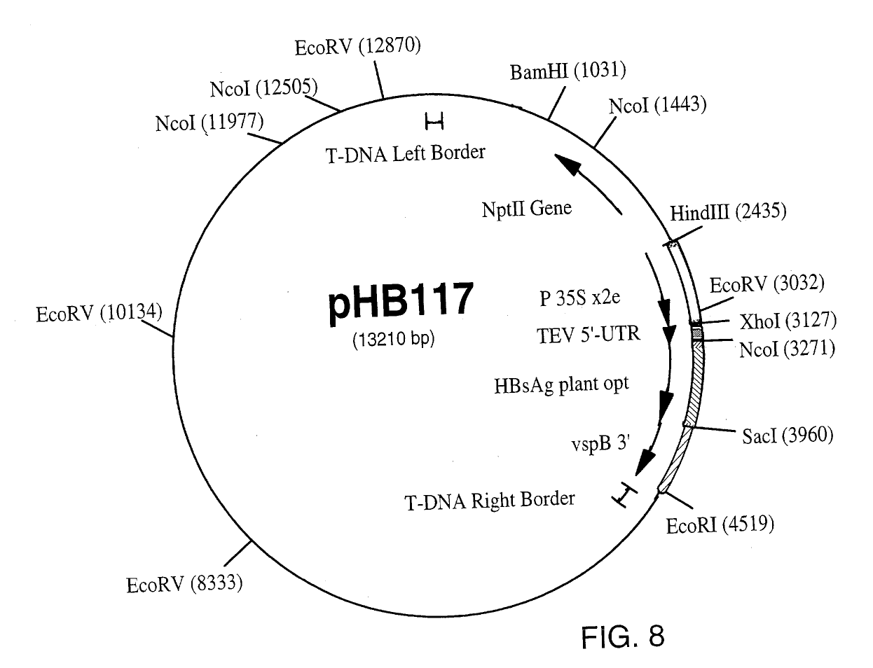

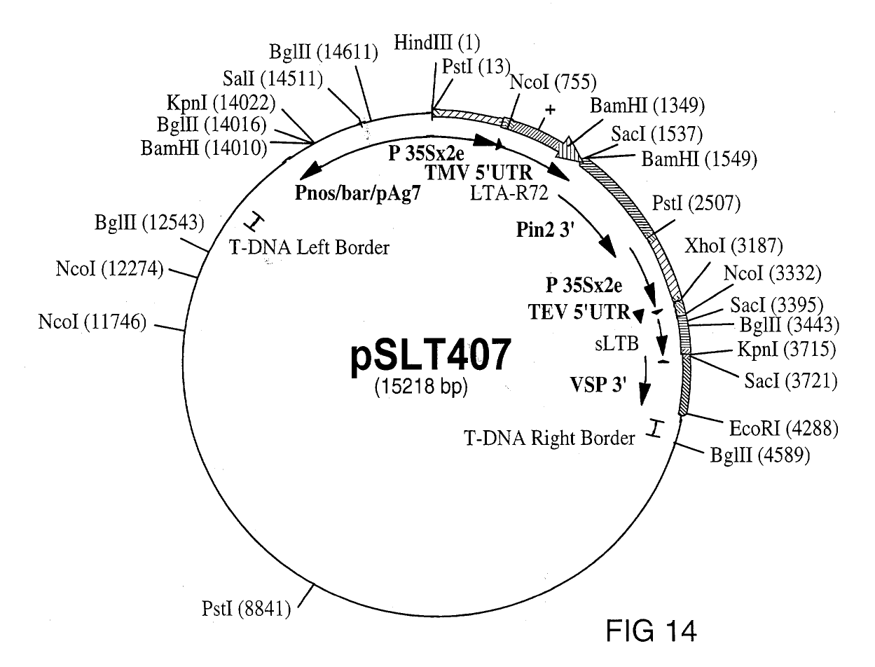

We created transgenic potato “Desiree” plants using pNV110 (FIG. 7) (for expression of Norwalk virus capsid protein, NVCP) and pHB 117 (FIG. 8) (for expression of hepatitis B surface antigen, HBsAg). These transgenic plants carry the selectable marker nptII for kanamycin resistance (kan^{R}). It is possible to “supertransfonn” these kan^{R} plants using a plasmid vector that contains an alternative selectable marker. We thus constructed pSLT407 (FIG. 14), which contains the selectable marker bar, encoding phosphinothricin acetyl trasferase and allowing selection on media containing phosphinothricin (PPT). pSLT407 also contains expression cassettes for the plant-optimized LT-A-R72 and the plant-optimized LT-B genes, and was constructed by ligation of the 4.1 kb HindIII/EcoRI fragment from pSLT107 with pGPTV-BAR (Becker D., et al., Plant Mol Biol 1992; 20:1195-1197).

Supertransformation of NV110 or HB117 transgenic lines with pSLT407 is performed exactly as described previously, except that the media used for regeneration of plantlets contains 1 mg/L PPT (Sigma Chemical Co., St. Louis, Mo.) and contain no kanamycin. Plantlets that form roots in medium containing 1 mg/L PPT are considered transgenic for the bar gene and thus the linked expression cassettes. Selected plantlets are assayed for expression of LT-B and assembled holotoxin LT-R72 by ganglioside-dependent ELISA as described in Example 9. Transformants that express LT-R72 at levels greater than 0.1% of total soluble leaf protein are then examined for expression of the antigen whose gene is linked to the nptII marker. Thus NV110/SLT407 plantlets are assayed for NVCP expression by ELISA (Mason et al, Proc. Natl. Acad. Sci. USA. 93: 5335-5340 (1996)); and HB117/SLT407 plantlets are assayed for HBsAg expression by ELISA. Mason et al., Proc. Natl. Acad Sci. USA. 89:11745-11749 (1992). It is necessary to verify that the insertion of the T-DNA from pSLT407 did not interfere with expression of the antigen whose gene is linked to the nptII marker; thus the levels of NVCP or HBsAg in the supertransformed plantlets should similar to those in the parent NV110 or HB117 plants.

NV110/SLT407 and H117/SLT407 lines are propagated, transplanted to soil, and grown in the greenhouse to maturity. Thus, tubers are harvested and examined as described previously for expression of LT-R72 and NVCP or HBsAg. Those lines that express LT-R72 at levels greater than 1 μg per g fresh tuber tissue and NVCP or HBsAg at levels greater than 10 μg per gram fresh tuber are selected for animal immunogenicity studies.

An alternative strategy for supertransformation to create NV110/SLT407 and HB117/SLT407 lines is to construct a bar vector (derivative of pGPTV-BAR) that contains the expression cassette for either NVCP or HBsAg. These are used to transform the transgenic lines SLT107 kan^{R} plants of Examples 10 and 14 in a similar manner. Plants regenerated and rooted on medium containing 1 mg/L PPT are assayed for NVCP expression by ELISA (Mason et al., 1996) or for HBsAg expression by ELISA (Mason et al., 1992). Selected plantlets are verified for LT-R72 expression and propagated for tuber production and assay as described above.

In order to assess the adjuvanticity of the LT-R72 co-delivered with the heterologous antigen, mice are fed 5 g doses of NV110/SLT407 tubers as described (Mason et al., 1996), and sera are tested by ELISA for antibodies against NVCP. A separate group of mice are fed NV110 tubers expressing similar levels of NVCP, and antibody levels in the two groups are compared for statistically significant differences.

### Example 16

### Tomato Transformation Protocol

Preparation of Plant Material:

Tomato seeds are sterilized by immersing them in a solution of 20% CHLOROX for twenty minutes, and then rinsing well two or more times with sterile milli-ω water. Approximately 30 sterilized seeds are sown in each Magenta box containing ½ MSO media (Tanksley TA234TM2R seed stock averages 100 seeds per 380 mg). The feeder layer is prepared by subculturing NT1 (Nicotiana Tobacum) weekly (2:48) in KCMS liquid medium. One day prior to cutting cotyledons, 2 mL from a one week old NT1 suspension culture is pipetted onto a KCMS-media plate. The suspension is covered with a sterile 7 cm Whatman filter and cultured overnight in a dark location. Eight days after sowing, each seedling is placed on a sterile paper towel moistened with sterile water, the cotyledon is excised at the petiole and the tips are cut off. Each sample is halved again if the size of the cotyledon is greater than 1 cm. The explants are laid adaxial side down on the prepared feeder plates and cultured overnight at 25° C., with a 16 hour photoperiod.

Transformation:

Agrobacterium is streaked onto LB selective media plates about 1 week prior to transformation and incubated 30° C. Liquid selective medium is inoculated by picking a single colony from the streaked plate, into 3 mL YM medium with 150 μg kanamycin sulfate. The liquid culture is then shaken vigorously at 30° C. for 48 hours. Using 250 mL flasks, 1 mL of inoculate is pipetted into 49 mL YM medium to which has been added 2.5 mg kanamycin sulfate. After shaking vigorously at 30° C. for 24 hours the culture O.D. is measured using a spectrophotometer at 600 nm, to obtain an optimum O.D. of 0.5-0.6.

The Agrobacterium culture is prepared for transformation by centrifuging at 8,000 rpm (Sorvall centrifuge, ss34 rotor) for 10 min. The YM medium is then poured off and the cell pellet resuspended in MS-0, 2%. The final O.D. should be between 0.5 and 0.6. The explants are incubated with the Agrobacterium culture/MS-0, 2% by pipetting 25 mL of culture into a sterile Magenta box, and transferring the explants from 2 to 3 plates into the same Magenta box. The explants are incubated for 5 minutes with occasional shaking, and then removed to a sterile paper towel. The explants are then returned adaxial side down to feeder plates, and the plates are sealed with Nesco film. The explants are cocultivated with a 16-hour photoperiod at 25° C. for approximately 24 hours, and then transferred adaxial side up to selection media (2Z). The plates are sealed with micropore tape and returned to 25° C. with a 16-hour photoperiod. The explants are transferred to new IZ selection medium plates every 3 weeks, and transferred to IZ Magenta boxes when shoots begin to appear.

Regeneration, Rooting and Selection:

Within 4 to 6 weeks, initial shoots appear. These shoots are excised from the explants when the shoots are at least 2 cm, being sure to include at least 1 node, and placed in Magenta boxes (4/box) containing Tomato Rooting Media with selective agents. New roots begin to appear in about 2 weeks.

Tomato line TA234 was transformed with pSLT103 described above, and several lines were screened that were rooting on kanamycin medium by Northern blot. Total RNA was prepared from tomato leaves, electrophoresed and blotted to nylon membranes as described (Mason et al, 1998). The blot was probed with a DNA fragment containing the sLT-A coding sequence, and those lines that showed the strongest signals by quantitation were selected using a Molecular Dynamics Phosphorimager. These lines were transplanted to soil in the greenhouse and grown to maturity.

### Example 17

### Holotoxin Expression in Tomato Transformed with pSLT103

This example shows that the K63 mutant form of LT-A can be expressed in fruits of transgenic tomato plants, and that it assembles with LT-B expressed in the same cells to produce a ganglioside-binding holotoxin complex. This observation illustrates that production of assembled A₁:B₅ holotoxin complexes can be achieved in edible fruits of whole plants.

Tomato (Lycopersicon esculentum L. cv. “TA234”) plants were stably transformed with pSLT103 which contains expression cassettes for mutant LT-A (K63) and LT-B. In this construct, the LT-B transcription is driven by the fruit-specific E8 promoter, such that no LT-B expression is expected in vegetative tissues. The LT-A-K63 cassette uses the constitutive CaMV 35S promoter, such that plantlets can be screened for expression by Northern blot of leaf RNA early during plantlet development.

The tomato plants were transformed by Agrobacterium-mediated DNA transfer as described above. Regenerated transgenic shoots were rooted on medium containing 50 μg/ml kanamycin. Leaves of plantlets propagated in agar medium were excised, and RNA was prepared as described (Mason et al., 1998). The RNA samples were assayed by Northern blot (data not shown), using the LT-A-K63 coding sequence as probe. The results show that expression varied greatly among independent transgenic lines, as expected for Agrobacterium-mediated DNA delivery. Line numbers 2, 10, 11, 12, 16, 17, and 23 were selected for propagation and transplanted them to soil for growth in the greenhouse.

### Example 18

### Holotoxin mLT-K63 Expression and Assembly in Fruit of Tomato Transformed with pSLT103

This example describes characterization of the mLT-K63 produced in fruits of ripening tomato plants transformed with pSLT103 as described in Example 17. The data show that assembled A₁B₅ complexes are assembled in the fruits, as determined by ganglioside-binding ELISA with antibody specific for LT-A.

Upon observation of flowers on the transgenic plants, pollen from these flowers was obtained and used to manually pollinate the stigmas to ensure self-fertilization and efficient fruit development. We then obtained fruit at varying stages of ripening, and extracted the fruits with 2 ml per gram fruit of buffer containing 50 mM sodium phosphate, pH 7.0, 100 mM NaCl, 1 mM EDTA, 1 mM PMSF, and 0.1% Triton-X-100, in a Ten-Broek ground-glass homogenizer (0.15 mm clearance). Extracts were centrifuged at 16,000×g for 3 minutes at 4° C., and dilutions of the extracts were assayed by ganglioside-dependent ELISA using anti-LT and anti-CT-A probes as described in Example 9, and for total soluble protein by the Bradford assay (BioRad kit) using BSA as a standard. Determinations of LT-B (anti-LT probe) and holo-LT (hLT) (anti-CT-A probe) were normalized to total soluble protein to obtain ng recombinant protein per μg total soluble protein, or normalized to sample fresh weight to obtain μg recombinant protein per g fresh fruit.

Because the E8 promoter that drives transcription of the LT-B gene in pSLT103 is active only during fruit ripening, we performed a study of transgenic fruits that were ripened to varying stages. For this purpose, the following definitions apply: mature green, fruit is filly enlarged but pigmentation is green with no trace of yellow or red; breaker, fruit begins to show yellow or orange pigmentation; pink, green pigmentation largely lost with orange to pink predominant; red, deep red pigmentation throughout the fruit, which is becoming soft to the touch. We assayed fruits of line SLT103-12 at different ripening stages by ELISA for LT-B or hLT. The results are shown in Table 8 below. No LT-B is seen in nontransgenic TA234 fruit or SLT103-12 fruit in mature green or breaker stages, but accumulates progressively in pink and red fruit. hLT-K63 is observed also in pink and red fruit, accounting for 22% and 12%, respectively, of the total LT-B. In other experiments hLT-K63 accounted for up to 30% of the total LT-B signal, and accumulated up to 2 μg per g of red tomato fruit. It is likely that some differences in cell type-specific expression occur for LT-B and LT-A-K63 since different promoters drive the two genes. Thus, a higher rate of assembly could be expected when the same promoter drives both genes.

TABLE 8
LT-B and hLT in fruits of SLT103-12 tomato plants

ng per μg TSP

| Line | Stage | LT-B | hLT | % Assembly |
|---|---|---|---|---|
| SLT103-12 | mature green | 0.0 | 0.0 | NA |
| SLT103-12 | breaker | 0.0 | 0.0 | NA |
| SLT103-12 | pink | 2.3 | 0.5 | 22% |
| SLT103-12 | red | 3.3 | 0.4 | 12% |
| TA234 | red | 0.0 | 0.0 | NA |

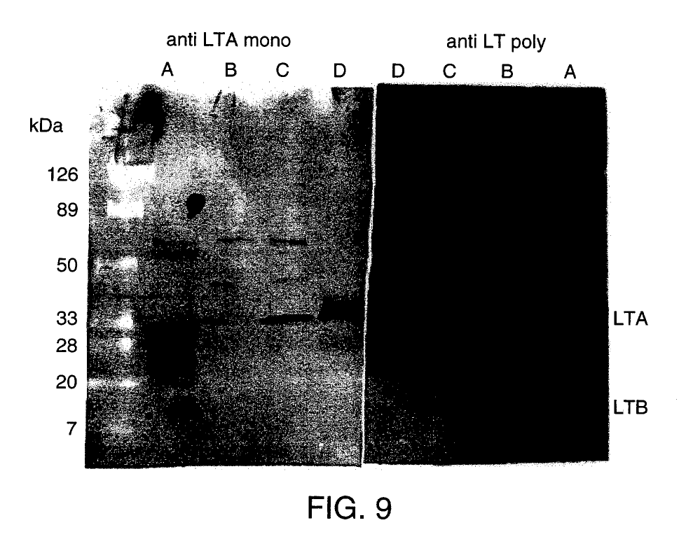

We performed Western blot analysis of the SLT103 tomato fruit using two different antibodies: a mouse monoclonal that is specific for LT-A (Chemicon, Temecula, Calif.), and a goat polyclonal antiserum that was raised against LT holotoxin (gift of John Clements, Tulane Medical Center, New Orleans, La.). The blot in FIG. 9 shows that transgenic plant SLT103-12 produces LT-A-K63 in fruit that co-migrates with the standard, bacterial LT-A-G192. This observation indicates that the plant material is correctly processed at the signal peptide cleavage site, but is not proteolytically processed at the trypsin cleavage site R192. In leaves of SLT103-12 plants, the LT-A-K63 protein appears to migrate slightly slower than that in fruit, suggesting that it may be at least partially glycosylated. A single N-linked glycosylation context occurs at N205 (N-L-S), suggesting that this site could be utilized for glycosylation in leaf cells. When a similar blot was probed with polyclonal anti-LT serum, LT-B was observed only in SLT103-12 fruit but not in leaf extracts, as expected. The fruit-derived LT-B signal co-migrated with bacterial LT-B, indicating that correct signal peptide processing, but no other posttranslational modifications occurred. Extract of nontransgenic TA234 leaf showed no LT-specific signal.

In conclusion, SLT103 transgenic tomato plants produce correctly processed A and B subunits of LT-K63 in ripe fruit. Further, the LT-B subunits efficiently assembled into ganglioside-binding pentamer form, accumulating up to approximately 7 μg per gram fruit. Assembled LT-K63 accumulated in these fruit at levels up to 2 μg per gram fruit. Thus, tomato is a potentially useful system for production of mutant LT forms for use as orally delivered mucosal adjuvant for co-delivered heterologous antigens, as well as a vaccine antigen for prevention of ETEC diarrhea.

### Example 19

### Tomato Plants transgenic for pSLT103 used for Sexual Crossing with Tomato Plants Transgenic for Heterologous Antigens

This example illustrates the use of a transgenic tomato line, SLT103-12, as described in Example 17, for sexual crossing with transgenic tomato plants that express heterologous antigens, in order to produce tomato lines that are transgenic for both pSLT103 and the heterologous construct and that produce both mLT-K63 and the heterologous antigen in fruit. These fruits would constitute an improved oral delivery system for the heterologous vaccine antigens, due to the benefit of co-presentation with the mucosal adjuvant mLT-K63.

We germinated seeds obtained from the self-fertilization of tomato line SLT103-12 and selected seedlings on agar medium containing 50 mg/L kanamycin. Thus seedlings that had lost the transgenes due to Mendelian segregation during meiosis and oocyte fertilization would not grow and would be eliminated from study. Kanamycin-resistant plants were transplanted to soil and grown in the greenhouse. Similarly, we germinated seeds of TA234 transgenic tomato lines NVT110 and HB117 and selected kanamycin-resistant seedlings. These lines were transgenic for pNVT110 (FIG. 7) (containing an expression cassette for Norwalk virus capsid protein, NVCP) and pHB117 (FIG. 8) (containing an expression cassette for hepatitis B surface antigen, HBsAg).

Transgenic plants were grown in the greenhouse until flowers appeared. Pollen was collected from flowers of SLT103-12 plants and applied to the stigmas of NVT110 and HB117 plants. Reciprocal crosses were performed by collection of pollen from flowers of NVT110 and HB117 plants, and application to stigmas of SLT103-12 flowers. Each flower that was the recipient of heterologous pollen was marked with a tag to identify the cross. The fruits are allowed to develop and ripen normally, and are harvested when pink to red. The progeny seeds are collected by straining fruits through a screen mesh, washed, and dried.

Progeny seeds are stored at 4° C. for 6 weeks before germination on agar medium containing 50 mg/L kanamycin. Kanamycin-resistant seedlings are then tested for presence of all appropriate transgenes by PCR of genomic DNA with gene-specific oligonucleotide primers. For example, the SLT103×NVT 110 cross is tested with primers specific for the coding sequences of plant-optimized LT-A-K63, plant-optimized LT-B, and NVCP. Seedlings that show positive PCR for all appropriate genes are grown to maturity in the greenhouse, and flowers are self-pollinated.

The fruits of the self-fertilized crosses are harvested when ripened to the pink to red stage, and assayed for mLT-K63 by ganglioside-dependent ELISA (see Example 9) and NVCP (Mason, H. et al., Proc. Natl. Acad. Sci. USA 1996; 93: 5335-5340) or HBsAg (Mason H. et al., Proc. Natl. Acad. Sci. USA 1992; 89:11745-11749) by the assays in cited works.

For immunogenicity assays, pink to red fruits of the progeny of appropriate crosses are fed directly to mice, approximately 10 g fresh fruit per dose. In control experiments, similar mice are fed the fruits expressing only the heterologous antigen (NVCP or HBsAg) without mLT-K63. Sera from the mice are collected at intervals after immunization and tested for presence of antibodies against the appropriate antigen (NVCP or HBsAg).

### Example 20

### Production of Rabbit Antiserum against LT-A-K63

This example describes production of rabbit antiserum specific for LT-A by immunization of rabbits with LT-A-K63 produced in E. coli. This specific antiserum is useful for detection of (mutant) LT-A subunits that are assembled with LT-B pentamers to form AIB₅ holotoxin complexes, using ganglioside-dependent ELISA. Because LT-B pentamers bind to ganglioside G_{M1} and unassembled LT-A subunits do not bind to ganglioside G_{M1}; this ELISA is a definitive assay for assembled holotoxin.

We obtained oligonucleotide primers:

sLTA-Bgl-F: (SEQ ID NO:57) 5′-CAATCCAAGGTGAAGAGGCAAAgaTctTCAGACTACCAATCAGAG-3′

sLTA-Bgl-R: (SEQ ID NO:58) 5′-CTCTGATTGGTAGTCTGAagAtcTTTGCCTCTTCACCTTGGATTG-3′

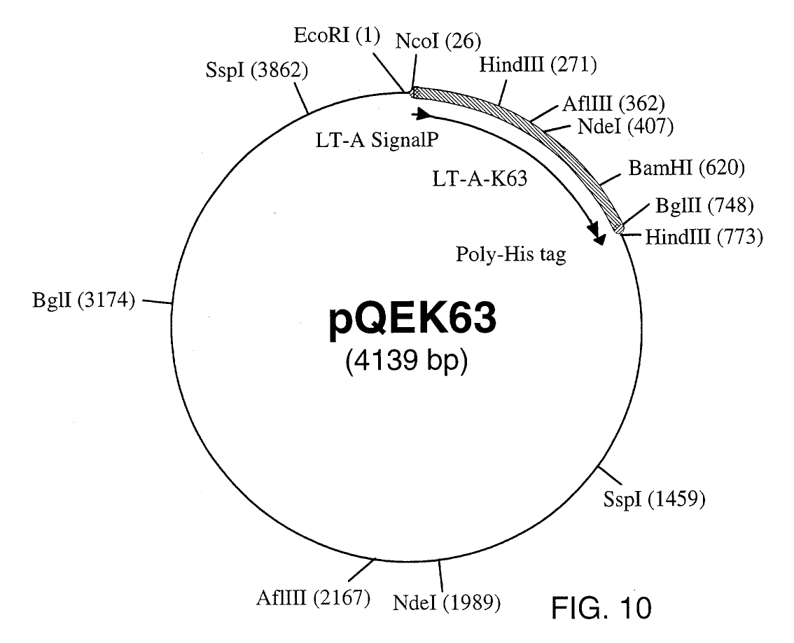

These primers anneal to create a BglII site immediately downstream of the Q121 of the mature LT-A. According to Sixma et al. (Nature 1991; 351:371-377) Q121 is the last residue of the A2 subunit that is not surrounded by the LT-B pentamer. We used these primers and the QUICKCHANGE™ site-directed mutagenesis kit (Strategene, La Jolla, Calif.) to create a BglII site in the LT-A-K63 gene contained in pSLTA(K63)-211 (Example 6). The mutation was confirmed by digestion with BglII and sequencing of 3′ region of gene. We then isolated the BstXI/BglII fragment from a confirmed mutant and ligated it along with the NcoI/BstXI fragment from pSLTA(K63)-211 (containing the 5′ end of the LT-A-K63 gene) into NcoI/BglII sites of pQE-60 (Qiagen) to form pQEK63 (FIG. 10). In this construct the LT-A-K63 fragment is fused to a 6-His tag at the C-terminus to facilitate purification of the recombinant protein by Ni²⁺-affinity chromatography.

E. coli BL21-(DE3) RIL cells (Stratagene, La Jolla, Calif.) were transformed with pQEK63. E. coli BL21-CodonPlus (DE3) RIL cells contain extra copies of the genes that encode tRNAs for codons in E. coli that are rarely used, as are found frequently in our plant-optimized LT-A-K63 gene. The pQEK63-transformed cells were grown at 37° C. overnight in 5ml LB liquid medium with 50 mg/L ampicillin. This culture was transferred to 100 ml LB liquid medium with 100 mg/L ampicillin and grown at 37° C. for two hours, at which point IPTG was added to a final concentration of 1 mM. The culture was grown for another four hours and cells were collected by centrifugation and frozen overnight at −20° C.

Cells were thawed for 15 minutes on ice then resuspended 5 ml per gram cell weight of lysis buffer (5 mM NaH₂PO₄, pH8.0; 300 mM NaCl; 0.1% Triton X-100; 20 mM β-mercaptoethanol, 1 mM PMSF; 10 mM imidazole). Cells were lysed via sonication (six 10-second bursts) and clarified by spinning at 10,000×g for 30 minutes at 4° C. Lysate was mixed with 1 ml of Ni-NTA matrix (Qiagen) for 1 hour on ice (with shaking). The slurry was then loaded onto a disposable minicolunm (BioRad) and allowed to drain. The resin was then washed twice with 4 ml of wash buffer (50 mM NaH₂PO₄, pH8.0; 300 mM NaCl; 1 mM PMSF; 20 mM imidazole) and eluted four times with 0.5 ml elution buffer (50 mM NaH₂PO₄, pH8.0; 300 mM NaCl; 1 mM PMSF; 250 mM imidazole). Samples were then analyzed on SDS-PAGE/PVDV blot followed by probing with anti-LT polyclonal goat serum (gift of Dr. John Clements, Tulane Medical Center, New Orleans, La.).

The eluted recombinant protein is dialyzed against PBS, pH 7.2 and used to immunize rabbits at 200 to 500 μg per dose. The first dose is emulsified with Freund's complete adjuvant, and second and third doses (1 and 2 months after the first dose) are emulsified with Freund's incomplete adjuvant. Antigen can be delivered subcutaneously at several sites on the back. Test bleeds 2 weeks after the second dose can be tested by ganglioside-dependent ELISA using mLT-G192 (gift of John Clements) or cholera toxin (CT) (Sigma Chemical Co.) as the standard. A similar ELISA using LT-B (gift of John Clements) or CT-B (Sigma Chemical Co.) as antigen is a negative control to verify that the antiserum does not cross-react with LT-B.

### Example 21

### Elicitation of an Immune Response in Mice and Chickens

Extracts of plant tissues expressing a polypeptide of the invention can be prepared by freezing the plant tissue in liquid nitrogen and homogenizing in a blender. The soluble protein can be extracted in buffer, 40 mM ascorbate, 20 mM EDTA, and 1 mM PMSF. The insoluble plant cell debris can be removed by centrifugation at 15,000×g. The plant extract can be precipitated with 40% ammonium sulfate at 4° C. The supernatant that is obtained after centrifugation at 15,000×g can be further precipitated at 60% ammonium sulfate final concentration at 4° C. This precipitate obtained after centrifugation at 15,000×g can be solubilized in PBS and used for oral immunization studies.

One group of mice can be given an extract of a plant extract or an intact plant orally. Another group of mice can be given a recombinant polypeptide purified from E. coli expressing the antigen from a recombinant plasmid. An antigen equivalent dose (as determined by ELISA) of for example, 5, 10, 12.5, 20, 50, or 100 μg can be administered to each mouse by gavage on for example, days 0, 4, 21, and 25. Serum and mucosal antibody responses can be examined by ELISA from samples collected on, for example days 30-32.

Isolation of serum and mucosal extracts from mice can be done by first anaesthetizing the mice with Xylazine/Ketamine. Serum can be removed by cardiac puncture and the mice sacrificed with cervical dislocation. The small intestine can be removed from the duodenum to cecum and placed in ice cold EDTA/STI (50 mM EDTA, 0.1 mg/ml soybean. trypsin inhibitor). The intestine can be homogenized in EDTA/STI using tissue homogenizer and then centrifuged at 32,000×g. The supernatant can be lyophilized overnight, resuspended and dialyzed in TEAN buffer, and assayed by ELISA essentially as described above and in example 9. The ELISA for IgA in mucosal material is similar to the ELISA for IgG, except that the material is probed with a dilution of goat antiserum-to mouse IgA (Sigma), which is in turn probed with a dilution of alkaline phosphatase conjugated antiserum to goat IgG (Sigma). See, e.g., WO 96/12801. The obtained values can be corrected for cross reactivity and contamination of the mucosa with serum. See WO 96/12801.

The ability of the serum and mucosal immunoglobulins obtained from animal immunized with the polypeptides of the invention can be tested for their ability to neutralize the biological activity of LT or CT to the same extent as serum and mucosal antibodies from animals immnunized orally with an antigen equivalent dose of bacterial produced recombinant polypeptides. Neutralization of adrenal cell assay can be conducted using mouse Y-1 adrenal cells in miniculture. A selected dose of toxin can be mixed with senral dilutions of pooled sera or mucosal samples from, for example, mice orally immunized with the extract of plant expressing a polypeptide of the invention or with a bacterial derived polypeptide. Following a preincubation for 1 hour at 37° C., samples can be applied to a monolayer of mouse YI adrenal cells (ATCC CCL79) and the incubation continued for 18 hours. Titer is defined as the reciprocal of the highest serum dilution showing complete neutralization of biological activity of, for example 50 picograms of toxin.

Chickens and other fowl comprise a sizable market for animal vaccines. As a test in fowl for the ability of food-borne antigens to elicit immune responses, chicks can be fed for example, raw potato tubers expressing an immunogenic polypeptide of the invention. The serum from these chicks can then be assayed for antibodies specific for the immunogenic polypeptide of the invention. For example, one day old Leghorn B12/B12 syngeneic chicks can be given tubers from transgenic potatoes containing approximately 5 μg of a polypeptide of the invention as assayed by ELISA. Five grams of the tubers can be fed to the chicks at for example, days 0, 4, 14, and 18. On for example, day 28, blood can be obtained from the chicks and serum prepared. The serum can be assayed by ELISA. See WO/96/12801.

### Example 22

### Dicistronic Cassette Vectors

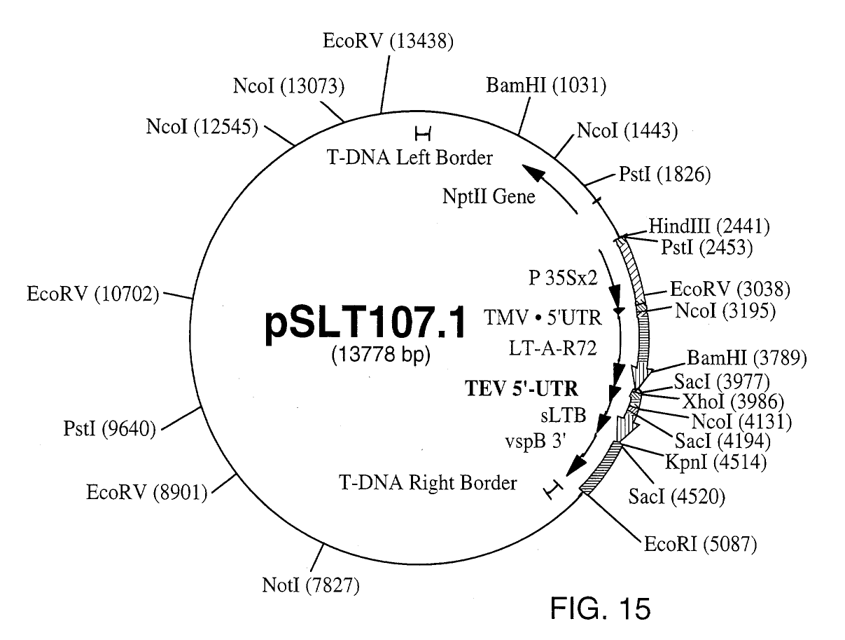

Polynucleotides of the invention can further be combined in an expression vector to form an expression cassette. For example, a plasmid can contain a dicistronic cassette for coexpression of an LT-A, CT-A, or mutants thereof, such as LT-A-R72, and an LT-B, CT-B, or mutants thereof, such as LT-B. An internal ribosome entry site (IRES) to can be used to stimulate translation initiation of the downstream LT-B or CT-B cistron. Any type of viral IRES element can be used, as referenced in Rohde W. et al., Plant viruses as model systems for the study of non-canonical translation mechanisms in higher plants. J. Gen. Virol., 1994; 75:2141-2149. Optionally, the tobacco etch virus 5′ UTR (Carrington J C & Freed D D. Cap-independent enhancement of translation by a plant polyvirus 5′ nontranslated region. J. Virol. 1990; 64:1590-1597), can be inserted between the LT-A or CT-A and the LT-B or CT-B polynucleotides. Preferably, the dicistronic cassette is driven by the 35S promoter and terminated by the vspB3′ region, but other promoters and termination regions can be used. FIG. 15 shows a plasmid with a dicistronic cassette for coexpression of LT-A-R72 and LT-B with an IRES sequence upstream of the LT-B polynucleotide. The tobacco etch virus 5′ UTR occurs between the polynucleotides and the dicistronic cassette is driven by the 35S promoter and terminated by the vspB3′region.

We claim:

53. A method of eliciting an immune response in an animal or human comprising the step of administering to an animal or human an immunogenic composition comprising a transgenic plant cell comprising a polynucleotide comprising a nucleic acid sequence encoding a mutant LT-A polypeptide or a mutant CT-A polypeptide, wherein the mutant LT-A or CT-A polypeptide has reduced enzyme activity as compared to a wild-type LT-A or CT-A polypeptide to a human or animal, wherein an immune response to at least one of said mutant LT-A or CT-A polypeptides is elicited, and wherein said CT-A polypeptide accumulates to higher levels in plants than do wild-type holotoxins or wild-type subunits.

54. The method of claim 53 wherein the composition is administered orally.

55. The method of claim 54 wherein the administration comprises consuming the transgenic plant cell.

58. The method of claim 53 further comprising administering an adjuvant wherein said adjuvant is mixed with said immunogenic composition.

59. The method of claim 53 wherein the immune response elicited is selected from the group of immune responses consisting of humoral; mucosal; cellular; humoral and mucosal; humoral and cellular; mucosal and cellular; and humoral, mucosal and cellular.
